SEQUENCE LISTING (200 sequences)

SEQ ID NO: 1 - DNA, 69 bp, Artificial Sequence
  Description of Artificial Sequence Synthetic oligonucleotide
ggtgtagctaaacgatagatagatagatagatagatagatagatagatagatagatattgtctgtgtac

SEQ ID NO: 2 - DNA, 48 bp, Artificial Sequence
  Description of Artificial Sequence Synthetic oligonucleotide
ggtgtagctaaacgatagatagatagatagatagatttgtctgtgtac

SEQ ID NO: 3 - DNA, 40 bp, Artificial Sequence
  Description of Artificial Sequence Synthetic oligonucleotide
gtacacagacaaatctatctatctatcgtttagctacacc

SEQ ID NO: 4 - DNA, 56 bp, Artificial Sequence
  Description of Artificial Sequence Synthetic oligonucleotide
gtacacagacaaatctatctatctatctatctatctatctatcgtttagctacacc

SEQ ID NO: 5 - DNA, 130 bp, Artificial Sequence
  Description of Artificial Sequence Synthetic oligonucleotide
cttcttcttcttctttcttcttcttcttcttcttcttcttcttcttcttcttcttcttcttcttcttcttcttcttcttcttcttcttcttcttcttcttcttcttctcctcctccttctccttcttctt

SEQ ID NO: 6 - DNA, 120 bp, Artificial Sequence
  Description of Artificial Sequence Synthetic oligonucleotide
ctccttctccttctccttctccttctcctacttcttcttcttcttcttcttcttcttcttcttcttcttcttcttcttcttcttcttcttcttcttcttcttcttctcctccttcttctt

SEQ ID NO: 7 - DNA, 35 bp, Artificial Sequence
  Description of Artificial Sequence Synthetic oligonucleotide
gagagagaagaagaaagagagaagaagaagaaaaa

SEQ ID NO: 8 - DNA, 81 bp, Artificial Sequence
  Description of Artificial Sequence Synthetic oligonucleotide
gaagaagaagaannnnnnnnnnnnnnnnnaggaggaggnnnnnnaggaggaggnnnnnnaggaggaggnnnnaggaggagg

SEQ ID NO: 9 - DNA, 116 bp, Artificial Sequence
  Description of Artificial Sequence Synthetic oligonucleotide
aagaagaagaagaagaagaagaagaagaagcaacagcagcagcagcagcagcagcagcagcagcagcggaggaggaggaggaggaggaggaggaggagaaaaaggagagagagaga

SEQ ID NO: 10 - DNA, 137 bp, Artificial Sequence
  Description of Artificial Sequence Synthetic oligonucleotide
ctttctttctttctttctttctttctttctttctttctttctttctttctttctttctttctttccttccttctttccttctttccttctttccttctttccttctttctttcttttctttctttctttctttcttt

SEQ ID NO: 11 - DNA, 88 bp, Artificial Sequence
  Description of Artificial Sequence Synthetic oligonucleotide
tctttctttcttttctttcttttctttcctttctttctttctttctttctttctttctttctttctttctttctttctttctttcttt

SEQ ID NO: 12 - DNA, 117 bp, Artificial Sequence
  Description of Artificial Sequence Synthetic oligonucleotide
ctttcctttcctttcctttcctttcctttcctttcctttcctttcctttcctttcctttccttccctccctccctccctccctccctcccttctttctctttctttctttctttctt

SEQ ID NO: 13 - DNA, 119 bp, Artificial Sequence
  Description of Artificial Sequence Synthetic oligonucleotide
tccttccttccttccttccttccttccttccttccttccttccttccttccttctttctttctttctttctttctttctttctttctttctttctttctttcttccttccttccttcct

SEQ ID NO: 14 - DNA, 72 bp, Artificial Sequence
  Description of Artificial Sequence Synthetic oligonucleotide
aaagaaagaaagaaaaagaaagaaagaaagaaagaaagaaagaaagaaagaaagaaagaaagaaagaaagaa

SEQ ID NO: 15 - DNA, 119 bp, Artificial Sequence
  Description of Artificial Sequence Synthetic oligonucleotide
ggaaggaaggaaggaaggaaggaaggaaggaaggaaagaaagaaagaaagaaagaaagaaagaaagaaagagaaagaaagaaagaaagaaagaaagaaagaaagaaagaaagaaagaaa

SEQ ID NO: 16 - DNA, 149 bp, Artificial Sequence
  Description of Artificial Sequence Synthetic oligonucleotide
tctttctttctttctttctttctttctttctttctttctttctttctttctttctttctttctttctttcttnnnnnnnnnnnnnnnntttttttctttctttctttctttctttctttctttctttctttctttctttctttcttttt

SEQ ID NO: 17 - DNA, 62 bp, Artificial Sequence
  Description of Artificial Sequence Synthetic oligonucleotide
tctttctttctttctttcttnnnnnnnnnnnnnnnntttttttattatttatttatttattt

SEQ ID NO: 18 - DNA, 80 bp, Artificial Sequence
  Description of Artificial Sequence Synthetic oligonucleotide
tttctttctttctttctttctttctttctttctttctttctttctttctttctttctttctctctctcctcctctttctt

SEQ ID NO: 19 - DNA, 85 bp, Artificial Sequence
  Description of Artificial Sequence Synthetic oligonucleotide
tccttctttctttctttnnnnnnnnnnnnctttctttctttctttctttctttctttctttctttctttctttctttctttcttt

SEQ ID NO: 20 - DNA, 119 bp, Artificial Sequence
  Description of Artificial Sequence Synthetic oligonucleotide
gagagnnnnnnnnnnnnnnnnnnnnnnnnnnnnnnnnngaaagaaagaaannnnnnnnnnnnnnnnnnnagaaagaaagaaagaaagaaagaaagaaagaaagaaagaaagaagagaag

SEQ ID NO: 21 - DNA, 112 bp, Artificial Sequence
  Description of Artificial Sequence Synthetic oligonucleotide
agagagaagagaagagaagagaagagaagagaagagaagagaagagaagagaagagaagagaagnnnnnnnnnnnnnnnnnnnnnnnnnnnnnnnngaaggaaggaaggaag

SEQ ID NO: 22 - DNA, 57 bp, Artificial Sequence
  Description of Artificial Sequence Synthetic oligonucleotide
aaaggaaaggaaaggaaaggnnnnnnnnnnnnnnnnaggggaggggaggggagggga

SEQ ID NO: 23 - DNA, 118 bp, Artificial Sequence
  Description of Artificial Sequence Synthetic oligonucleotide
gggaagggaagggaagggaaaagaaaggaaaggaaaggaaaggaaaggaaaggaaaggaaaggaaaggaaaggaaaggaaaggaaaggaaaggaaaggaaaggaaaggaaaggaaagg

SEQ ID NO: 24 - DNA, 38 bp, Artificial Sequence
  Description of Artificial Sequence Synthetic oligonucleotide
agaaagaaagaaaggaaaaaagaaagaaagagagagag

SEQ ID NO: 25 - DNA, 101 bp, Artificial Sequence
  Description of Artificial Sequence Synthetic oligonucleotide
agaaagaaagaaagaaagaaagaaagaaagaaagaaagaaagaaagaaagaaagaaagaaagaaagaaagaaagaaaaagagaaagagagagggagggagg

SEQ ID NO: 26 - DNA, 83 bp, Artificial Sequence
  Description of Artificial Sequence Synthetic oligonucleotide
cttcttcttcttcttcttcttcttcttcttcttcttcttcttcttcttcttcttcttcttcttcttcttttcttcttcttctt

SEQ ID NO: 27 - DNA, 137 bp, Artificial Sequence
  Description of Artificial Sequence Synthetic oligonucleotide
ccttccttccttccttccttccttccttccttccttccttccttccttccttccttccttccttccttccttnnnnnnnccctccctccctnnnnnctctctctnnnnnnnnnnnnnnnnnnnnnctctctctctct

SEQ ID NO: 28 - DNA, 254 bp, Artificial Sequence
  Description of Artificial Sequence Synthetic oligonucleotide
ccattccattccattnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnattccattccattccattccattccattccattccattccattccattccattccnnnnnnnnnnnnnnnnnnnnnnnnnnnnnnnnnnnattccattccattccattccattccattccattccattccattccattcc

SEQ ID NO: 29 - DNA, 113 bp, Artificial Sequence
  Description of Artificial Sequence Synthetic oligonucleotide
gaaaggaaaggaaaggaaaggaaaggaaaggaaaggaagggaagggaagggaagggaagggaagggaagggaagggaagggaagggaagggaagggaagggaagggaagggaa

SEQ ID NO: 30 - DNA, 156 bp, Artificial Sequence
  Description of Artificial Sequence Synthetic oligonucleotide
agagatagagatagagatagagatagagatagagatagagatagagatagagatagagatagagatnnnnnnnnnnagagatagagatagagatnnnnnnnnnnnnnnagagatagagatagagatagagatagagatagagatagagatagagat

SEQ ID NO: 31 - DNA, 91 bp, Artificial Sequence
  Description of Artificial Sequence Synthetic oligonucleotide
atagatagatgatagatagatagatagatagatagatagatagatagatagatacatacatacatacatacatacatacatacatacatac

SEQ ID NO: 32 - DNA, 66 bp, Artificial Sequence
  Description of Artificial Sequence Synthetic oligonucleotide
tactactactactactactactactactactactactattattattgttattattattattattat

SEQ ID NO: 33 - DNA, 82 bp, Artificial Sequence
  Description of Artificial Sequence Synthetic oligonucleotide
gaaagaaagaaannnnnngaaagaaagaaagaaagaaagaaagaaagaaagaaagaaagaaagaaagaaagaaagaaagaaa

SEQ ID NO: 34 - DNA, 113 bp, Artificial Sequence
  Description of Artificial Sequence Synthetic oligonucleotide
tccctttccctttccctttccctttccctttccctttccctttccctttccctttccctttccctttccctttccctcccctcccctcccctcccctctcctttcctttcctt

SEQ ID NO: 35 - DNA, 68 bp, Artificial Sequence
  Description of Artificial Sequence Synthetic oligonucleotide
tttctttctttctttctttctttctttctttctttctttctttctttctttctttctttctttctttc

SEQ ID NO: 36 - DNA, 68 bp, Artificial Sequence
  Description of Artificial Sequence Synthetic oligonucleotide
aaagaaagaaagaaagaaagaaagaaagaaagaaagaaagaaagaaagaaagaaagaaagaaagaaag

SEQ ID NO: 37 - DNA, 101 bp, Artificial Sequence
  Description of Artificial Sequence Synthetic oligonucleotide
aaaggaaaggaaaggaaaggaaaggnnnnnnnnnnnnnnnnnnnnnnnnnngaaaagaaaagaaaagaaaagaaaagaaaagaaaagaaaagaaaagaaaa

SEQ ID NO: 38 - DNA, 56 bp, Artificial Sequence
  Description of Artificial Sequence Synthetic oligonucleotide
tccttccttccttccttccttccttccttccttccttccttccttccttccttcct

SEQ ID NO: 39 - DNA, 39 bp, Artificial Sequence
  Description of Artificial Sequence Synthetic oligonucleotide
ataataataataataataataataataataataataata

SEQ ID NO: 40 - DNA, 52 bp, Artificial Sequence
  Description of Artificial Sequence Synthetic oligonucleotide
agatagatagatagatagatagatagatagatagatagatagatagatagat

SEQ ID NO: 41 - DNA, 39 bp, Artificial Sequence
  Description of Artificial Sequence Synthetic oligonucleotide
attattattattattattattattattattattattatt

SEQ ID NO: 42 - DNA, 60 bp, Artificial Sequence
  Description of Artificial Sequence Synthetic oligonucleotide
ttttattttattttattttattttattttattttattttattttattttattttatttta

SEQ ID NO: 43 - DNA, 44 bp, Artificial Sequence
  Description of Artificial Sequence Synthetic oligonucleotide
aaataaataaataaataaataaataaataaataaataaataaat

SEQ ID NO: 44 - DNA, 44 bp, Artificial Sequence; identical to SEQ ID NO: 43
  Description of Artificial Sequence Synthetic oligonucleotide

SEQ ID NO: 45 - DNA, 44 bp, Artificial Sequence
  Description of Artificial Sequence Synthetic oligonucleotide
ttatttatttatttatttatttatttatttatttatttatttat

SEQ ID NO: 46 - DNA, 44 bp, Artificial Sequence; identical to SEQ ID NO: 43
  Description of Artificial Sequence Synthetic oligonucleotide

SEQ ID NO: 47 - DNA, 33 bp, Artificial Sequence
  Description of Artificial Sequence Synthetic oligonucleotide
ataataataataataataataataataataata

SEQ ID NO: 48 - DNA, 33 bp, Artificial Sequence
  Description of Artificial Sequence Synthetic oligonucleotide
aacaacaacaacaacaacaacaacaacaacaac

SEQ ID NO: 49 - DNA, 44 bp, Artificial Sequence
  Description of Artificial Sequence Synthetic oligonucleotide
atttatttatttatttatttatttatttatttatttatttattt

SEQ ID NO: 50 - DNA, 44 bp, Artificial Sequence; identical to SEQ ID NO: 43
  Description of Artificial Sequence Synthetic oligonucleotide

SEQ ID NO: 51 - DNA, 44 bp, Artificial Sequence; identical to SEQ ID NO: 43
  Description of Artificial Sequence Synthetic oligonucleotide

SEQ ID NO: 52 - DNA, 44 bp, Artificial Sequence; identical to SEQ ID NO: 49
  Description of Artificial Sequence Synthetic oligonucleotide

SEQ ID NO: 53 - DNA, 40 bp, Artificial Sequence
  Description of Artificial Sequence Synthetic oligonucleotide
aataaataaataaataaataaataaataaataaataaata

SEQ ID NO: 54 - DNA, 40 bp, Artificial Sequence
  Description of Artificial Sequence Synthetic oligonucleotide
tgtttgtttgtttgtttgtttgtttgtttgtttgtttgtt

SEQ ID NO: 55 - DNA, 30 bp, Artificial Sequence
  Description of Artificial Sequence Synthetic oligonucleotide
aacaacaacaacaacaacaacaacaacaac

SEQ ID NO: 56 - DNA, 40 bp, Artificial Sequence
  Description of Artificial Sequence Synthetic oligonucleotide
aaataaataaataaataaataaataaataaataaataaat

SEQ ID NO: 57 - DNA, 30 bp, Artificial Sequence
  Description of Artificial Sequence Synthetic oligonucleotide
aataataataataataataataataataat

SEQ ID NO: 58 - DNA, 36 bp, Artificial Sequence
  Description of Artificial Sequence Synthetic oligonucleotide
tatttatttatttatttatttatttatttatttatt

SEQ ID NO: 59 - DNA, 27 bp, Artificial Sequence
  Description of Artificial Sequence Synthetic oligonucleotide
tgttgttgttgttgttgttgttgttgt

SEQ ID NO: 60 - DNA, 36 bp, Artificial Sequence
  Description of Artificial Sequence Synthetic oligonucleotide
aataaataaataaataaataaataaataaataaata

SEQ ID NO: 61 - DNA, 36 bp, Artificial Sequence
  Description of Artificial Sequence Synthetic oligonucleotide
ctttctttctttctttctttctttctttctttcttt

SEQ ID NO: 62 - DNA, 40 bp, Artificial Sequence
  Description of Artificial Sequence Synthetic oligonucleotide
aaacaaaacaaaacaaaacaaaacaaaacaaaacaaaaca

SEQ ID NO: 63 - DNA, 40 bp, Artificial Sequence
  Description of Artificial Sequence Synthetic oligonucleotide
ttttgttttgttttgttttgttttgttttgttttgttttg

SEQ ID NO: 64 - DNA, 24 bp, Artificial Sequence
  Description of Artificial Sequence Synthetic oligonucleotide
ttattattattattattattatta

SEQ ID NO: 65 - DNA, 40 bp, Artificial Sequence
  Description of Artificial Sequence Synthetic oligonucleotide
gttttgttttgttttgttttgttttgttttgttttgtttt

SEQ ID NO: 66 - DNA, 48 bp, Artificial Sequence
  Description of Artificial Sequence Synthetic oligonucleotide
tatatctatatctatatctatatctatatctatatctatatatatata

SEQ ID NO: 67 - DNA, 21 bp, Artificial Sequence
  Description of Artificial Sequence Synthetic oligonucleotide
aacaacaacaacaacaacaac

SEQ ID NO: 68 - DNA, 28 bp, Artificial Sequence
  Description of Artificial Sequence Synthetic oligonucleotide
aaacaaacaaacaaacaaacaaacaaac

SEQ ID NO: 69 - DNA, 35 bp, Artificial Sequence
  Description of Artificial Sequence Synthetic oligonucleotide
ttttgttttgttttgttttgttttgttttgttttg

SEQ ID NO: 70 - DNA, 21 bp, Artificial Sequence
  Description of Artificial Sequence Synthetic oligonucleotide
ttgttgttgttgttgttgttg

SEQ ID NO: 71 - DNA, 24 bp, Artificial Sequence
  Description of Artificial Sequence Synthetic oligonucleotide
aaataaataaataaataaataaat

SEQ ID NO: 72 - DNA, 20 bp, Artificial Sequence
  Description of Artificial Sequence Synthetic oligonucleotide
attgattgattgattgattg

SEQ ID NO: 73 - DNA, 25 bp, Artificial Sequence
  Description of Artificial Sequence Synthetic oligonucleotide
aaaacaaaacaaaacaaaacaaaac

SEQ ID NO: 74 - DNA, 30 bp, Artificial Sequence
  Description of Artificial Sequence Synthetic oligonucleotide
ccacctccacctccacctccacctccacct

SEQ ID NO: 75 - DNA, 25 bp, Artificial Sequence
  Description of Artificial Sequence Synthetic oligonucleotide
tttgttttgttttgttttgttttgt

SEQ ID NO: 76 - DNA, 20 bp, Artificial Sequence
  Description of Artificial Sequence Synthetic oligonucleotide
aaaacaaaacaaaacaaaac

SEQ ID NO: 77 - DNA, 20 bp, Artificial Sequence
  Description of Artificial Sequence Synthetic primer
agccacctgggtatatgagg

SEQ ID NO: 78 - DNA, 20 bp, Artificial Sequence
  Description of Artificial Sequence Synthetic primer
tgttgcagcttttccttctg

SEQ ID NO: 79 - DNA, 22 bp, Artificial Sequence
  Description of Artificial Sequence Synthetic primer
ggcattatgtgtttgtgagtgc

SEQ ID NO: 80 - DNA, 20 bp, Artificial Sequence
  Description of Artificial Sequence Synthetic primer
acagactggcaaccaaaagg

SEQ ID NO: 81 - DNA, 20 bp, Artificial Sequence
  Description of Artificial Sequence Synthetic primer
aggttgggttgtgtcaacag

SEQ ID NO: 82 - DNA, 21 bp, Artificial Sequence
  Description of Artificial Sequence Synthetic primer
agcaggacttcagcaagagag

SEQ ID NO: 83 - DNA, 20 bp, Artificial Sequence
  Description of Artificial Sequence Synthetic primer
atccccaaaatctgaaatgc

SEQ ID NO: 84 - DNA, 22 bp, Artificial Sequence
  Description of Artificial Sequence Synthetic primer
aactgccagctgaacataaaac

SEQ ID NO: 85 - DNA, 21 bp, Artificial Sequence
  Description of Artificial Sequence Synthetic primer
accagaagttaaaggctgtgg

SEQ ID NO: 86 - DNA, 21 bp, Artificial Sequence
  Description of Artificial Sequence Synthetic primer
cctggatgatgaactgtaggg

SEQ ID NO: 87 - DNA, 19 bp, Artificial Sequence
  Description of Artificial Sequence Synthetic primer
gccagcagtagacccagac

SEQ ID NO: 88 - DNA, 20 bp, Artificial Sequence
  Description of Artificial Sequence Synthetic primer
tgaggcaggaaaatcacttg

SEQ ID NO: 89 - DNA, 20 bp, Artificial Sequence
  Description of Artificial Sequence Synthetic primer
caccactgtgccaagctatt

SEQ ID NO: 90 - DNA, 20 bp, Artificial Sequence
  Description of Artificial Sequence Synthetic primer
cagagcaaccctctgtcaag

SEQ ID NO: 91 - DNA, 19 bp, Artificial Sequence
  Description of Artificial Sequence Synthetic primer
tgggaaactgatccaaacc

SEQ ID NO: 92 - DNA, 19 bp, Artificial Sequence
  Description of Artificial Sequence Synthetic primer
gggttacttcgccagaagg

SEQ ID NO: 93 - DNA, 20 bp, Artificial Sequence
  Description of Artificial Sequence Synthetic primer
aaaccatccttgcatcacag

SEQ ID NO: 94 - DNA, 21 bp, Artificial Sequence
  Description of Artificial Sequence Synthetic primer
ccaaaaccagacaaacacctc

SEQ ID NO: 95 - DNA, 20 bp, Artificial Sequence
  Description of Artificial Sequence Synthetic primer
aatggagattggacatgctg

SEQ ID NO: 96 - DNA, 23 bp, Artificial Sequence
  Description of Artificial Sequence Synthetic primer
cagttgaaggtaaagcaaaatcc

SEQ ID NO: 97 - DNA, 20 bp, Artificial Sequence
  Description of Artificial Sequence Synthetic primer
gtgactggagaactgctgga

SEQ ID NO: 98 - DNA, 22 bp, Artificial Sequence
  Description of Artificial Sequence Synthetic primer
ttccttttggttttatgccttt

SEQ ID NO: 99 - DNA, 20 bp, Artificial Sequence
  Description of Artificial Sequence Synthetic primer
ttgtgctcatgtaccctgga

SEQ ID NO: 100 - DNA, 20 bp, Artificial Sequence
  Description of Artificial Sequence Synthetic primer
cctcctgtctgccattttgt

SEQ ID NO: 101 - DNA, 20 bp, Artificial Sequence
  Description of Artificial Sequence Synthetic primer
gttgtccggctttttgagtt

SEQ ID NO: 102 - DNA, 20 bp, Artificial Sequence
  Description of Artificial Sequence Synthetic primer
ctcccacaggaagaagaagg

SEQ ID NO: 103 - DNA, 20 bp, Artificial Sequence
  Description of Artificial Sequence Synthetic primer
ttgcttgtacccagaagacg

SEQ ID NO: 104 - DNA, 20 bp, Artificial Sequence
  Description of Artificial Sequence Synthetic primer
aggaattggacccctcaatc

SEQ ID NO: 105 - DNA, 19 bp, Artificial Sequence
  Description of Artificial Sequence Synthetic primer
ttgcagtaagcggagatcg

SEQ ID NO: 106 - DNA, 20 bp, Artificial Sequence
  Description of Artificial Sequence Synthetic primer
aaatggaaagcaaaccttgg

SEQ ID NO: 107 - DNA, 20 bp, Artificial Sequence
  Description of Artificial Sequence Synthetic primer
tgaggcaggagaatagcttg

SEQ ID NO: 108 - DNA, 20 bp, Artificial Sequence
  Description of Artificial Sequence Synthetic primer
tgagagacttcccactccac

SEQ ID NO: 109 - DNA, 20 bp, Artificial Sequence
  Description of Artificial Sequence Synthetic primer
ggaaggggaacatcacactc

SEQ ID NO: 110 - DNA, 20 bp, Artificial Sequence
  Description of Artificial Sequence Synthetic primer
acggtctcaatctcctgacc

SEQ ID NO: 111 - DNA, 20 bp, Artificial Sequence
  Description of Artificial Sequence Synthetic primer
ctgttctgctgttggctgac

SEQ ID NO: 112 - DNA, 20 bp, Artificial Sequence
  Description of Artificial Sequence Synthetic primer
acatggtaaaaccccgtctc

SEQ ID NO: 113 - DNA, 20 bp, Artificial Sequence
  Description of Artificial Sequence Synthetic primer
gaaatcctggctgtgtcctc

SEQ ID NO: 114 - DNA, 20 bp, Artificial Sequence
  Description of Artificial Sequence Synthetic primer
tctaagggatgcaaggtgtg

SEQ ID NO: 115 - DNA, 20 bp, Artificial Sequence
  Description of Artificial Sequence Synthetic primer
ctaagcccacaaggtcaagg

SEQ ID NO: 116 - DNA, 20 bp, Artificial Sequence
  Description of Artificial Sequence Synthetic primer
cattcagcagccagtgattc

SEQ ID NO: 117 - DNA, 20 bp, Artificial Sequence
  Description of Artificial Sequence Synthetic primer
agagtgcccttgtattgcag

SEQ ID NO: 118 - DNA, 20 bp, Artificial Sequence
  Description of Artificial Sequence Synthetic primer
ctgaggcaggagaattgttg

SEQ ID NO: 119 - DNA, 20 bp, Artificial Sequence
  Description of Artificial Sequence Synthetic primer
catgttacccacctctcctg

SEQ ID NO: 120 - DNA, 20 bp, Artificial Sequence
  Description of Artificial Sequence Synthetic primer
gcagcactccaaaatgacag

SEQ ID NO: 121 - DNA, 20 bp, Artificial Sequence
  Description of Artificial Sequence Synthetic primer
gggttgctttctgctaggtg

SEQ ID NO: 122 - DNA, 20 bp, Artificial Sequence
  Description of Artificial Sequence Synthetic primer
tggtgtgcttctcttccttc

SEQ ID NO: 123 - DNA, 20 bp, Artificial Sequence
  Description of Artificial Sequence Synthetic primer
ctggcattcaaatcctctcc

SEQ ID NO: 124 - DNA, 20 bp, Artificial Sequence
  Description of Artificial Sequence Synthetic primer
cagtgtctcttcctgggttg

SEQ ID NO: 125 - DNA, 22 bp, Artificial Sequence
  Description of Artificial Sequence Synthetic primer
aaagaagagaagcacaccacac

SEQ ID NO: 126 - DNA, 21 bp, Artificial Sequence
  Description of Artificial Sequence Synthetic primer
gtcccaatatgatggaagagg

SEQ ID NO: 127 - DNA, 20 bp, Artificial Sequence
  Description of Artificial Sequence Synthetic primer
ctcccacagaaacacacacc

SEQ ID NO: 128 - DNA, 20 bp, Artificial Sequence
  Description of Artificial Sequence Synthetic primer
acccagtgaaaacccatctc

SEQ ID NO: 129 - DNA, 20 bp, Artificial Sequence
  Description of Artificial Sequence Synthetic primer
gaagtgcgtgtcctcaccta

SEQ ID NO: 130 - DNA, 21 bp, Artificial Sequence
  Description of Artificial Sequence Synthetic primer
tttgtttccctctccttctca

SEQ ID NO: 131 - DNA, 20 bp, Artificial Sequence
  Description of Artificial Sequence Synthetic primer
agcacagataatgccactgc

SEQ ID NO: 132 - DNA, 20 bp, Artificial Sequence
  Description of Artificial Sequence Synthetic primer
tctccttcgttccttccttc

SEQ ID NO: 133 - DNA, 20 bp, Artificial Sequence
  Description of Artificial Sequence Synthetic primer
ggcaagatgaacaaggtgtc

SEQ ID NO: 134 - DNA, 20 bp, Artificial Sequence
  Description of Artificial Sequence Synthetic primer
actggagggaaccaactctg

SEQ ID NO: 135 - DNA, 20 bp, Artificial Sequence
  Description of Artificial Sequence Synthetic primer
attgggtgacaacactccag

SEQ ID NO: 136 - DNA, 20 bp, Artificial Sequence
  Description of Artificial Sequence Synthetic primer
gtaagcgtgggaaaacaatg

SEQ ID NO: 137 - DNA, 21 bp, Artificial Sequence
  Description of Artificial Sequence Synthetic primer
tcaggagaaaattccaaaagc

SEQ ID NO: 138 - DNA, 20 bp, Artificial Sequence
  Description of Artificial Sequence Synthetic primer
cagtgagccaagatggtgac

SEQ ID NO: 139 - DNA, 20 bp, Artificial Sequence
  Description of Artificial Sequence Synthetic primer
ggggtagtggggaaggatag

SEQ ID NO: 140 - DNA, 20 bp, Artificial Sequence
  Description of Artificial Sequence Synthetic primer
ccaggcaatagagcaagacc

SEQ ID NO: 141 - DNA, 25 bp, Artificial Sequence
  Description of Artificial Sequence Synthetic primer
gaatatcctagctgtgaatctcctc

SEQ ID NO: 142 - DNA, 19 bp, Artificial Sequence
  Description of Artificial Sequence Synthetic primer
catgggaaaaacccaacac

SEQ ID NO: 143 - DNA, 20 bp, Artificial Sequence
  Description of Artificial Sequence Synthetic primer
cctgggcaacagagaaagac

SEQ ID NO: 144 - DNA, 20 bp, Artificial Sequence
  Description of Artificial Sequence Synthetic primer
caccacacctggctaagaag

SEQ ID NO: 145 - DNA, 20 bp, Artificial Sequence
  Description of Artificial Sequence Synthetic primer
tggtgaaatcccgtctctac

SEQ ID NO: 146 - DNA, 20 bp, Artificial Sequence
  Description of Artificial Sequence Synthetic primer
ttcttggggaaggtatcagc

SEQ ID NO: 147 - DNA, 20 bp, Artificial Sequence
  Description of Artificial Sequence Synthetic primer
aaaccacaaaagcacattcc

SEQ ID NO: 148 - DNA, 20 bp, Artificial Sequence
  Description of Artificial Sequence Synthetic primer
atgagaatcgcttgaacctg

SEQ ID NO: 149 - DNA, 20 bp, Artificial Sequence
  Description of Artificial Sequence Synthetic primer
aggtggtttgatttgctttg

SEQ ID NO: 150 - DNA, 20 bp, Artificial Sequence
  Description of Artificial Sequence Synthetic primer
tcaagaggctgaggaaagag

SEQ ID NO: 151 - DNA, 25 bp, Artificial Sequence
  Description of Artificial Sequence Synthetic primer
tgatggatagaaacacagaaataca

SEQ ID NO: 152 - DNA, 20 bp, Artificial Sequence
  Description of Artificial Sequence Synthetic primer
ttacaaccctgcaaaggaag

SEQ ID NO: 153 - DNA, 20 bp, Artificial Sequence
  Description of Artificial Sequence Synthetic primer
aggcagaggttccagtaagc

SEQ ID NO: 154 - DNA, 21 bp, Artificial Sequence
  Description of Artificial Sequence Synthetic primer
ggatgctgggtcaaacagtag

SEQ ID NO: 155 - DNA, 21 bp, Artificial Sequence
  Description of Artificial Sequence Synthetic primer
agaactttggcagactttgtg

SEQ ID NO: 156 - DNA, 20 bp, Artificial Sequence
  Description of Artificial Sequence Synthetic primer
aggtgggaggattgtttgag

SEQ ID NO: 157 - DNA, 21 bp, Artificial Sequence
  Description of Artificial Sequence Synthetic primer
tgagaaaagtctcgccttacc

SEQ ID NO: 158 - DNA, 20 bp, Artificial Sequence
  Description of Artificial Sequence Synthetic primer
gaggcatgaggttgtgtgac

SEQ ID NO: 159 - DNA, 22 bp, Artificial Sequence
  Description of Artificial Sequence Synthetic primer
cctaaaagttacaacccagcac

SEQ ID NO: 160 - DNA, 20 bp, Artificial Sequence
  Description of Artificial Sequence Synthetic primer
gcctggtgacagagtgagac

SEQ ID NO: 161 - DNA, 22 bp, Artificial Sequence
  Description of Artificial Sequence Synthetic primer
gaggggagtgtagaaagaatgc

SEQ ID NO: 162 - DNA, 19 bp, Artificial Sequence
  Description of Artificial Sequence Synthetic primer
agggggctgagtaatggag

SEQ ID NO: 163 - DNA, 22 bp, Artificial Sequence
  Description of Artificial Sequence Synthetic primer
ccaaataaactgtggatggaag

SEQ ID NO: 164 - DNA, 20 bp, Artificial Sequence
  Description of Artificial Sequence Synthetic primer
gcaacagggggaatactctg

SEQ ID NO: 165 - DNA, 20 bp, Artificial Sequence
  Description of Artificial Sequence Synthetic primer
ctgcccaacatagtgaaacc

SEQ ID NO: 166 - DNA, 21 bp, Artificial Sequence
  Description of Artificial Sequence Synthetic primer
gagattacaggcaccaccatc

SEQ ID NO: 167 - DNA, 19 bp, Artificial Sequence
  Description of Artificial Sequence Synthetic primer
ctgggtgtgcattcgagac

SEQ ID NO: 168 - DNA, 20 bp, Artificial Sequence
  Description of Artificial Sequence Synthetic primer
cctgggtgacagactccatc

SEQ ID NO: 169 - DNA, 20 bp, Artificial Sequence
  Description of Artificial Sequence Synthetic primer
gctgcctctaatgtgagctg

SEQ ID NO: 170 - DNA, 20 bp, Artificial Sequence
  Description of Artificial Sequence Synthetic primer
aggatggtctcgatttcctg

SEQ ID NO: 171 - DNA, 20 bp, Artificial Sequence
  Description of Artificial Sequence Synthetic primer
actgtcaccccttgactgag

SEQ ID NO: 172 - DNA, 20 bp, Artificial Sequence
  Description of Artificial Sequence Synthetic primer
gaagctgaggcaggagaaag

SEQ ID NO: 173 - DNA, 20 bp, Artificial Sequence
  Description of Artificial Sequence Synthetic primer
acttgggtgggtgttactgg

SEQ ID NO: 174 - DNA, 20 bp, Artificial Sequence
  Description of Artificial Sequence Synthetic primer
tgaggataatgggcaaaatg

SEQ ID NO: 175 - DNA, 25 bp, Artificial Sequence
  Description of Artificial Sequence Synthetic primer
tttttcttgctctttttcttttctc

SEQ ID NO: 176 - DNA, 20 bp, Artificial Sequence
  Description of Artificial Sequence Synthetic primer
ttgcaccattgcactctagg

SEQ ID NO: 177 - DNA, 20 bp, Artificial Sequence
  Description of Artificial Sequence Synthetic primer
gcagtaggaaggctggagac

SEQ ID NO: 178 - DNA, 20 bp, Artificial Sequence
  Description of Artificial Sequence Synthetic primer
ttctttggccctgcatttac

SEQ ID NO: 179 - DNA, 22 bp, Artificial Sequence
  Description of Artificial Sequence Synthetic primer
gaaattgtgacataccgctgac

SEQ ID NO: 180 - DNA, 20 bp, Artificial Sequence
  Description of Artificial Sequence Synthetic primer
cgagcaacagtgcaagactc

SEQ ID NO: 181 - DNA, 23 bp, Artificial Sequence
  Description of Artificial Sequence Synthetic primer
tggctgtactctattccaggttc

SEQ ID NO: 182 - DNA, 20 bp, Artificial Sequence
  Description of Artificial Sequence Synthetic primer
tgacgagttagtgggtgcag

SEQ ID NO: 183 - DNA, 20 bp, Artificial Sequence
  Description of Artificial Sequence Synthetic primer
catgcacctgtaatcccaac

SEQ ID NO: 184 - DNA, 20 bp, Artificial Sequence
  Description of Artificial Sequence Synthetic primer
cttcaccctcaaaagcaatg

SEQ ID NO: 185 - DNA, 21 bp, Artificial Sequence
  Description of Artificial Sequence Synthetic primer
ggtgggttttagttggctatg

SEQ ID NO: 186 - DNA, 20 bp, Artificial Sequence
  Description of Artificial Sequence Synthetic primer
ttcttgattccctgtgttgg

SEQ ID NO: 187 - DNA, 21 bp, Artificial Sequence
  Description of Artificial Sequence Synthetic primer
tttttctgtgggtctgaatcc

SEQ ID NO: 188 - DNA, 21 bp, Artificial Sequence
  Description of Artificial Sequence Synthetic primer
cctgggagatgtctgtttttc

SEQ ID NO: 189 - DNA, 20 bp, Artificial Sequence
  Description of Artificial Sequence Synthetic primer
agagtctagggcgacagagc

SEQ ID NO: 190 - DNA, 21 bp, Artificial Sequence
  Description of Artificial Sequence Synthetic primer
tgccatttagattgtggtttg

SEQ ID NO: 191 - DNA, 20 bp, Artificial Sequence
  Description of Artificial Sequence Synthetic primer
tgcttgaaccttggagacag

SEQ ID NO: 192 - DNA, 20 bp, Artificial Sequence
  Description of Artificial Sequence Synthetic primer
ttgacttgttgaccctgtgg

SEQ ID NO: 193 - DNA, 21 bp, Artificial Sequence
  Description of Artificial Sequence Synthetic primer
gctgagattactggtgtgagc

SEQ ID NO: 194 - DNA, 20 bp, Artificial Sequence
  Description of Artificial Sequence Synthetic primer
catgttgctgggagtgagac

SEQ ID NO: 195 - DNA, 20 bp, Artificial Sequence
  Description of Artificial Sequence Synthetic primer
ctctccagcttttcccactg

SEQ ID NO: 196 - DNA, 20 bp, Artificial Sequence
  Description of Artificial Sequence Synthetic primer
gggcaccattttcaggatag

SEQ ID NO: 197 - DNA, 20 bp, Artificial Sequence
  Description of Artificial Sequence Synthetic primer
ggttgaggtgggagaatagc

SEQ ID NO: 198 - DNA, 20 bp, Artificial Sequence
  Description of Artificial Sequence Synthetic primer
ggcccagtagcaatacagtg

SEQ ID NO: 199 - DNA, 20 bp, Artificial Sequence
  Description of Artificial Sequence Synthetic primer
attatatcccgtccgattcc

SEQ ID NO: 200 - DNA, 19 bp, Artificial Sequence
  Description of Artificial Sequence Synthetic primer
ttggtgtgaactggagtgg

(19) United States
(12) Patent Application Publication
(10) Pub. No.: US 2009/0117542 A1
(43) Pub. Date: May 7, 2009

(54) UNIQUE SHORT TANDEM REPEATS AND METHODS OF THEIR USE

(75) Inventors: Julie L. Maybruck, Alexandria, VA (US); Paul A. Fuerst, Columbus, OH (US)

Correspondence Address: CALFEE HALTER & GRISWOLD, LLP, 800 SUPERIOR AVENUE, SUITE 1400, CLEVELAND, OH, 44114, US

(73) Assignee: The Ohio State University Research Foundation, Columbus, OH (US)

(21) Appl. No.: 11/569,324

(22) PCT Filed: May 16, 2005
(86) PCT No.: PCT/US05/17137

§ 371 Date: Nov. 19, 2007

Related U.S. Application Data
(60) Provisional application No. 60/571,825, filed on May 17, 2004.

(51) Int. Cl. C12Q 1/68; C07H 21/04

(52) U.S. Cl. 435/6; 536/24.33

(57) ABSTRACT

Methods for DNA fingerprinting identification of human DNA samples, comprising: a) exposing a DNA sample of an individual to at least one primer specific for a Y chromosome polymorphism at a predetermined loci, said loci being chosen from OSU9, OSU14, OSU22, OSU35, OSU51, OSU57, OSU67, OSU70, OSU73, OSU77, with the proviso that if OSU70 is selected then at least one other OSU locus is also selected; b) amplifying DNA of the DNA sample using the at least one primer specific for a Y chromosome polymorphism; and c) identifying the size of an amplified product. Primers for the methods are also provided.

### DESCRIPTION OF THE INVENTION

This application claims priority to U.S. Provisional Application No. 60/571,825, filed May 17, 2004, the entire disclosure of which is incorporated herein by reference.

### FIELD OF THE INVENTION

The invention relates to short tandem repeats of nucleotide sequences in a genome. The collection of short tandem repeats of this invention can be used, for example, to identify relationships between and within populations, trace migration routes, exclude individuals as suspects in crimes, and identifying paternity and maternity.

### BACKGROUND OF THE INVENTION

Short Tandem Repeats (STRs) found in genomic nucleotide sequences have proven to be highly informative markers in medical genetics, population genetics, and forensics. STRs are variable genetic markers found throughout the genome. The most widely used STRs are 2-7 base pair repeated sequences. FIG. 1 depicts an example of an STR locus. Primer sequences are designed from the unique sequence surrounding the repeat, which generally ensures the amplification of one locus. An exception to this occurs when the primer sequences are duplicated elsewhere in the genome resulting in the amplification of additional products. Allelic differences are due to the number of repeats in the repeat stretch (FIG. 1).

Allelic changes occur during replication and are caused by replication slippage (FIG. 2). It has been hypothesized that mutations in STRs occur according to the stepwise mutation model. This model suggests that allele changes occur most frequently with the addition or removal of one repeat at a time. In general, loci with fewer repeat stretches increase in size and loci with longer repeat stretches decrease in size (Wierdl et al., 1997; Schlotterer, 2000).

Short Tandem Repeats are presently the preferred genetic markers in DNA forensics. They are extremely informative due to the high degree of variability between individuals. In addition to the many applications of STRs in forensic science, they are also useful in population studies. Together with mitochondrial DNA, Y-STRs allow the examination of both maternal and paternal migration patterns of the same populations (Hurles et al., 1998; Perez-Lezaun et al., 1999). Thus, STRs are useful in identifying relationships between and within populations, tracing migration routes, excluding individuals as suspects in crimes, and identifying paternity and maternity.

### SUMMARY OF THE INVENTION

While other groups have introduced/characterized new loci on the Y-chromosome for forensic purposes (Kayser et al., 1997; White et al., 1999; Ayub et al., 2000; lida et al., 2001; lida et al., 2002; Redd et al., 2002; Kayser et al. 2004) (see Table 1 and FIG. 3), the loci identified by these groups lack the desired specificity. Thus, improvements can be made. The present invention presents a novel collection of short tandem repeats.

TABLE 1

| Loci | Period Size | Literature Source |
|---|---|---|
| DYS19 | Tetranucleotide | Arnemann et al., 1985 |
| DXYS156Y | Pentanucleotide | Chen et al., 1994 |
| YCAI, YCAII, YCAIII | Dinucleotide | Mathias et al., 1994 |
| G10123 | Trinucleotide | Murray et al. GDB 1995 |
| DYF371, DYS425 (One of the DYF371 loci), | Trinucleotide | Jobling et al., 1996 |
| DYS426 | | |
| DYS385, DYS389 I &II, DYS390, DYS391, | Tetranucleotide | Kayser et al., 1997 |
| DYS392, DYS393 | | |
| DYS388 | Trinucleotide | |
| DYS288 | Dinucleotide | |
| Y-GATA-A4, Y-GATA-A7.1 (DYS 460), Y-GATA- | Tetranucleotide | White et al., 1999 |
| A7.2 (DYS461), Y-GATA-A8, Y-GATA-A10, Y- | | |
| GATA-C4, Y-GATA-H4 | | |
| DYS438 | Pentanucleotide | Ayub et al., 2000 |
| DYS434, DYS435, DYS437, DYS439 | Tetranucleotide | |
| DYS436 | Trinucleotide | |
| DYS441, DYS442 | Tetranucleotide | Iida et al., 2001 |
| DYS443, DYS444, DYS445 | Tetranucleotide | Iida et al., 2002 |
| DYS462 | Tetranucleotide | Bosch et al., 2002 |
| DYS448 | Hexanucleotide | Redd et al., 2002 |
| DYS446, DYS447, DYS450, DYS452, DYS463 | Pentanucleotide | |
| DYS449, DYS453, DYS454, DYS455, DYS456, | Tetranucleotide | |
| DYS458, DYS459, DYS464 | | |
| DYS594, DYS589, DYS643 | Pentanucleotide | Kayser et al., 2004 |
| DYF406S1, DYS505, DYS508, DYS522, DYS525, | Tetranucleotide | |
| DYS531, DYS533, DYS540, DYS549, DYS556, | | |
| DYS570, DYS575, DYS576, DYS578, DYS636 | | |
| DYS638, DYS641 | | |
| DYS485, DYS488, DYS490, DYS494, DYS495, | Trinucleotide | |
| DYS617 | | |

The invention provides DNA amplification primer pairs for the amplification of at least one short tandem repeat marker, wherein the primer pair is chosen from the primer pairs listed in Table 4. In some embodiments, the primer pair is chosen from the primer pairs corresponding to the loci listed in Table 5.

The invention also provides a method for DNA fingerprinting at least one genetically related or unrelated individual, comprising: a) exposing a DNA sample of an individual to at least one primer specific for a Y chromosome polymorphism at a predetermined locus, said locus being chosen from those listed in Table 2, with the proviso that if OSU70 is selected then at least one other locus from Table 2 is also selected; b) amplifying DNA of the DNA sample using the at least one primer specific for a Y chromosome polymorphism; and c) identifying the size of an amplified product. In some embodiments, the DNA amplification of step b) is effected by PCR or by asymmetric PCR procedure. In some embodiments, the amplifying is performed using a primer pair as described above.

The invention also relates to methods for DNA fingerprinting identification of human DNA samples, comprising: a) exposing a DNA sample of an individual to at least one primer specific for a Y chromosome polymorphism at a predetermined locus, said locus being chosen from OSU9, OSU14, OSU22, OSU35, OSU51, OSU57, OSU67, OSU70, OSU73, OSU77, with the proviso that if OSU70 is selected then at least one other OSU locus is also selected; b) amplifying DNA of the DNA sample using the at least one primer specific for a Y chromosome polymorphism; and c) identifying the size of an amplified product. In some embodiments, the DNA fingerprinting of said DNA samples is for verifying transplanted tissues in research or therapeutic procedures. In some embodiments, the DNA fingerprinting of said DNA samples is for single cell genetic profiling in research or therapeutic procedure. In some embodiments, the DNA fingerprinting of said DNA samples is for verifying sample mix-up or contamination. In some embodiments, the DNA fingerprinting of said DNA samples is for testing, establishing or verifying paternity, maternity or consanguinity of individuals.

The invention also relates to kits for amplification of Y chromosomal polymorphisms, comprising: at least one primer pair as described; at least one reagent necessary for carrying out DNA amplification; and at least one component that makes it possible to determine length of an amplified fragment.

The invention also provides methods for determining the degree of relatedness between two or more individuals having the same or a different surname, comprising: a) obtaining a DNA sample from said individuals; b) amplifying said DNA by polymerase chain reaction using primers specific for Y chromosome polymorphisms at predetermined loci, said loci being selected from the group consisting of OSU9, OSU14, OSU22, OSU35, OSU51, OSU57, OSU67, OSU70, OSU73, OSU77, with the proviso that if OSU70 is selected then at least one other OSU locus is also selected; c) determining the haplotypes of said individuals; and d) comparing said haplotypes across a plurality of predetermined loci to determine the degree of relatedness between said individuals. In some embodiments, the DNA sample is isolated from a source selected from the group consisting of blood cells, fingernail slices, and hair follicles.

Additional objects and advantages of the invention will be set forth in part in the description that follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate one (several) embodiment(s) of the invention and together with the description, serve to explain the principles of the invention.

### BRIEF DESCRIPTION OF THE DRAWINGS

FIG. 1 shows an example of a tetranucleotide short tandem repeat. GATA, denoted in gray and underlined, is the repeat or period size. The repeat stretch for this allele is 11 GATAs. The unique sequence surrounding the repeat is the sequence from which primers can be designed.

FIG. 2 shows how mutation in STRs occurs through replication slippage. In this Figure, allele numbers are altered by two repeat stretches. The gray sequence denotes the GATA/CTAT repeat. * represents the newly synthesized strand strands. a) Original sequence with five GATA/CTAT repeats. b) Replication slippage reducing the repeat stretch. The template strand has folded on itself and the two GATA repeats are not copied in the newly synthesized strand, reducing the number of repeats by two. c) Replication slippage increasing the repeat stretch. The newly synthesized strand has folded on itself and the two GATA repeats are copied an additional time, increasing the number of repeats by two.

FIG. 3 shows chromosomal localization of some previously identified loci. The majority of listed loci occur in two small regions of the Y-chromosome. The loci in black were identified prior to the identification of the present loci. YCAII is the only dinucleotide repeat presented, since it is in the extended haplotype in the Y-STR databases. The gray loci are the loci identified by other researches during the course of this study.

FIG. 4 shows chromosomal localization of new loci. Sixty-two new loci were identified using the human genome sequence. They are present in regions outside that of the previously available loci. The unlabeled gray horizontal lines represent the most widely used previously available loci identified prior to the onset of this study (Kayser et al., 1997; White et al., 1999; Ayub et al., 2002). The vertical lines adjacent to the ruler are the six contigs annotated in GenBank that were analyzed in the study.

FIG. 5 shows chromosomal localization of the 10-locus set. Ten of the 62 loci were chosen that were the most appropriate for forensic purposes. As in FIG. 4, the unlabeled gray horizontal lines represent the previously available loci identified prior to the onset of this study (Kayser et al., 1997; White et al., 1999; Ayub et al., 2002). The vertical lines adjacent to the ruler are the six contigs annotated in GenBank analyzed in the study.

FIG. 6a) OSU73, b) OSU9 and c) OSU57 are examples of nine of the 10 loci that exhibit different allelic distributions in Caucasian and African American populations. FIG. 6d) OSU51 is the only locus that did not show a significantly different allelic distribution for the two populations. All alleles seen in the 30-individual population are represented.

FIG. 7 shows Y-chromosome homology. The majority of the duplicated regions are found on the X- or Y-chromosome. The Y-chromosome is represented on the left whereas the X-chromosome is on the right. The three columns from left to right represent the general regions of homology, identified in this study, with the autosomes, Y-, and X-chromosomes, respectively. Several of the loci, duplicated on the X- or Y-chromosome, were also found to be duplicated on autosomes. One major and six minor regions were found that are duplicated on the X-chromosome. The major region is in the p arm of the Y-chromosome in 11.2 proximal to the telomeres while the duplicated region on the X-chromosome is in 21.2 and 21.31 proximal to the centromeric region on the q arm. The 1^{st} minor region on the Y-chromosome is also located in the p arm in 11.31 proximal to the telomeric region and is found on the X-chromosome proximal to the telomeric region of the p arm in 22.22. The 2^{nd} minor region is situated just below the major region on the p arm of the Y-chromosome in 11.2 and just above the major region on the X-chromosome in the q arm in 21.1. The 3^{rd} minor region is found midway through the p arm on the Y-chromosome in 11.2 and is proximal to the telomeric region on the X-chromosome in the p arm in 22.33. The 4^{th} minor region is midway through the p arm of the Y-chromosome in 11.2 and is positioned on the X-chromosome proximal to the telomeric region on the q arm in 27.1. The 5^{th} minor region rests proximal to the centromeric region in 11.2 in the p arm of the Y-chromosome and nearly midway through the p arm on the X-chromosome in 21.3. The 6^{th} minor region is proximal to the telomeric region of the q arm on the Y-chromosome in 12 and proximal to the telomeric region in the q arm on the X-chromosome in 28.

FIG. 8 shows the distribution of alleles for OSU-10 locus and Y-PLEX sets (collected from Reliagene's Y-PLEX™ 6 and Y-PLEX™ 5 sets). A comparison of the number of alleles present in the same 30 individuals using the OSU 10-locus set.

FIG. 9 shows allelic distribution for all 30 individuals in the Y-PLEX 10-locus set. a) DYS19; b) DYS385; c) DYS3891; d) DYS38911; e) DYS390; f) DYS391; g) DYS392; h) DYS393; i) DYS438; j) DYS439.

FIG. 10 shows allelic distribution for all 30 individuals in the OSU 10-locus set. a) OSU9; b) OSU14; c) OSU22; d) OSU35; e) OSU51; f) OSU57; g) OSU67; h) OSU70; i) OSU73; j) OSU77.

FIG. 11 shows the distribution of the number of pairwise allelic differences between haplotypes. FIG. 11a) is the Y-PLEX 10-locus set and FIG. 11b) is the OSU 10-locus set.

FIG. 12 shows a bubble plot of pairwise haplotype comparisons between each of 30 individuals utilizing either the Y-PLEX or the OSU 10-locus sets. (Each individual was compared with every other individual.) X-axis and Y-axis show the number of allelic differences between pairs of individuals for the Y-PLEX 10 and OSU 10-locus sets, respectively. Dotted line indicates the diagonal, where both kits give equal number of differences. Data is skewed toward greater differences with the OSU 10-locus set.

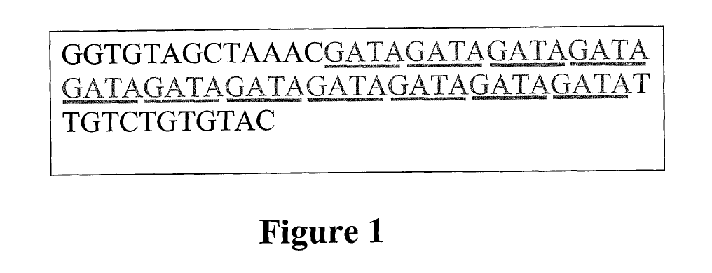

### DESCRIPTION OF THE EMBODIMENTS

The present invention will now be described by reference to more detailed embodiments, with occasional reference to the accompanying drawings. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, the term “contig” means a list or diagram showing an ordered arrangement of cloned overlapping fragments that collectively contain the sequence of an originally continuous DNA strand.

The present invention is directed to methods and kits for identifying individual primates, including humans, through the use of a novel collection of short tandem repeats (STRs). The methods and kits of the invention can be used to identify relationships between and within populations, trace migration routes, exclude individuals as suspects in crimes, and identify paternity and maternity.

In one embodiment of the invention, the methods comprise assaying at least one biological sample from a primate (e.g., human) subject for the presence of at least one short tandem repeat (STR) marker in the Y-chromosome DNA of the subject, wherein the at least one STR marker is chosen from the loci listed in Table 4. In some embodiments, the STR markers are chosen from the OSU 10-Locus Set listed in Table 5: OSU9, OSU14, OSU22, OSU35, OSU51, OSU57, OSU67, OSU70, OSU73, OSU77, with the proviso that if OSU70 is selected then at least one other OSU locus is also selected. In some embodiments, more than one, two, three, four, five, six, seven, eight, or nine, or more, loci are selected for use in the assay or kit.

The presence of the loci listed in Table 4 and 5 can be identified using the primer pairs listed in Table 4. These primer pairs, and kits containing them, are also within the scope of the invention. Thus, primer pairs can be chosen from those listed in Table 4; in some embodiments, the primer pairs are chosen from those for identifying OSU9, OSU14, OSU22, OSU35, OSU51, OSU57, OSU67, OSU70, OSU73, and OSU77. The invention is also directed to isolated and/or purified nucleotide sequences complementary to, or that hybridize under stringent conditions with, the primer pairs of this invention.

In some embodiments of the present invention, a data-mining element is included, whereby large amounts of data are subjected to an analytic process that searches for systematic relationships between particular features. Each derived pattern can be tested against new data sets until a robust model is identified.

The biological sample that is tested according to this invention may be any sample that contains nucleic acid material, such as DNA. Such samples can include, for example, nucleated cellular material. Samples include, but are not limited to, blood, sweat, saliva, semen, and any other primate bodily component in any amount. Various methods can be used to release the nucleic acid material from its surrounding tissue or cellular material so that it can be more effectively assayed or tested. Such separation methods are well known in the art.

In some embodiments of the invention, assaying involves a nucleic acid amplification step. Examples of such methods are well known in the art, and include, for example, the polymerase chain reaction (PCR). Briefly, in this process, the double strand of the DNA molecule is disrupted by a heating process. Polymerase enzymes and nucleic acid substrates are provided to encourage a new complementary strand to develop and bind with the single stranded molecule chain as the reaction mix cools. Each time the process is repeated the amount of DNA is amplified. The amplification becomes limited when the enzymes and substrates are exhausted.

Particular regions of the DNA molecule are developed by introducing short sequences of DNA that are complementary to and adjacent to the area of interest on the molecule, such that these will readily bind to the single stranded molecule as it cools, providing an enabling start to the production of the second strand. Later detection of these areas of interest within the molecule is facilitated with some form of detectable label, such as a fluorescent marker, which can be introduced into the manufactured primer sequence.

Thus, this invention includes, for example, methods for detecting the presence of at least one STR in a biological sample, comprising: a) bringing the biological sample into contact with a pair of oligonucleotide primers as described above, the DNA contained in the sample having been optionally made available to hybridization and under conditions permitting a hybridization of the primers with the DNA contained in the biological sample; b) amplifying the DNA; c) revealing the amplification products; and d) detecting the presence of the STR.

Step d) of the above-described method may comprise a single-strand conformation polymorphism (SSCP); a denaturing gradient gel electrophoresis (DGGE); sequencing (Smith, L. M., Sanders, J. Z., Kaiser, R. J., Fluorescence detection in automated DNA sequence analysis. Nature 1986; 321:674-9); a molecule hybridization capture probe or a temperature gradient gel electrophoresis (TGGE).

Step c) of the above-described method may comprise the detection of the amplified products with an oligonucleotide probe as defined above.

In one embodiment, the invention comprises: a) bringing the biological sample into contact with an oligonucleotide probe according to the invention, the DNA contained in the sample having been optionally made available to hybridization and under conditions permitting a hybridization of the primers with the DNA contained in the biological sample; and b) detecting the hybrid formed between the oligonucleotide probe and the DNA contained in the biological sample. This step may comprise single-strand conformation polymorphism (SSCP), a denaturing gradient gel electrophoresis (DGGE), or amplification and sequencing.

The invention also includes kits for the detection of particular STRs, comprising: a) a pair of oligonucleotide primers according to the invention; b) the reagents necessary for carrying out DNA amplification; and

c) a component that makes it possible to determine the length of the amplified fragments or to detect a mutation.

### EXAMPLES

### Example 1

### Identification of New Y-chromosome Short Tandem Repeat (Y-STR) Loci

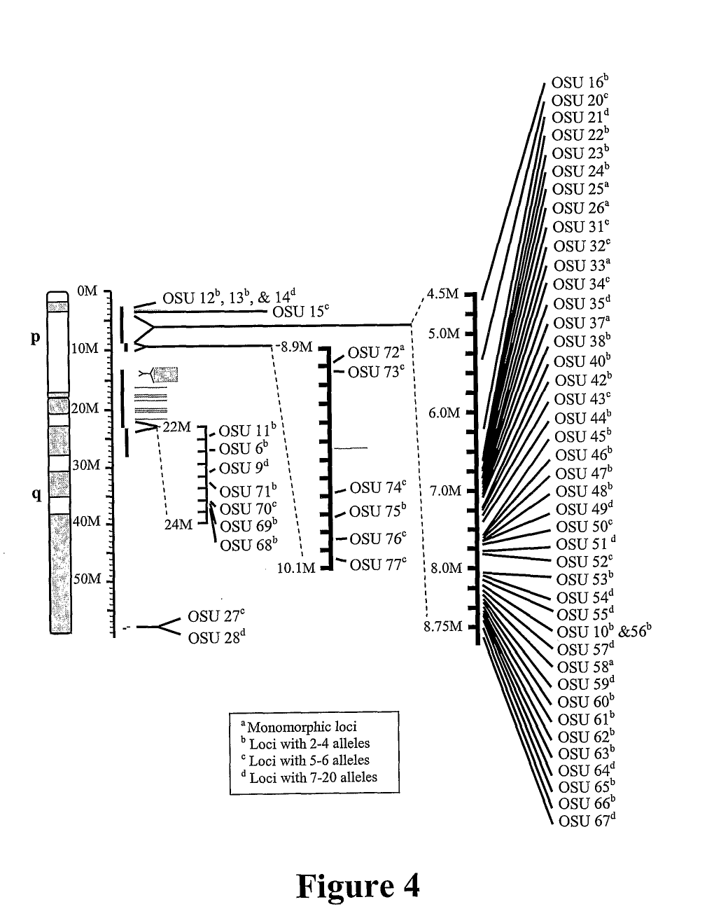

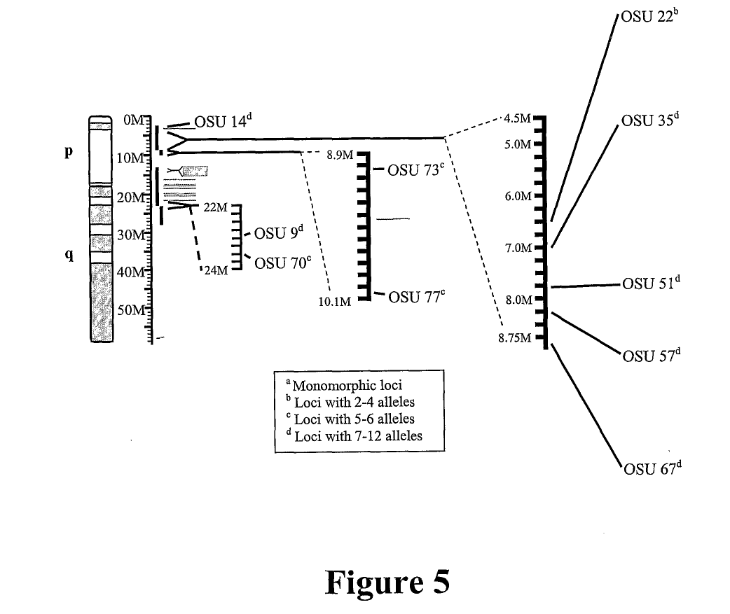

Briefly, this Example describes the identification of 62 new loci that span the length of 23 Mb of the annotated region of the Y-chromosome (FIG. 4). The loci were screened in a population of 30 racially diverse individuals to determine the number of alleles associated with each locus (Table 3). From the present 62 loci, a subset of 10 loci (FIG. 5) was chosen that were male-specific, distributed along the Y-chromosome outside of the regions with high concentrations of loci, and contained the most polymorphic loci in the regions of interest. Seven of the 62 loci, and one of the 10 loci, are identical to those published by Redd et al. in 2002.

Materials and Methods

Microsatellite Identification

DNA sequences were retrieved from the draft version of the Human Genome Project. Due to the contingent nature of the Y-chromosome genomic sequence, locations of sequences of interest had to be confirmed multiple times since the onset of the study. The Y-chromosome sequence consists of approximately 59 Mb. Presently, nearly 26 Mb have been annotated and released in the public database of the National Center for Biotechnology Information (GenBank).

Using the sequence from the public database, 63 potential Y-STR loci located in regions not previously represented were identified. The computer program “Tandem Repeats Finder” (http://tandem.bu.edu/trf/trf.html) (Benson, 1999) was used to identify the STRs. The output included 200 base pairs of flanking sequence on either side of each repeat. Primers were designed from the flanking sequence using the computer program, Primer3 (http://frodo.wi.mit.edu/primer3/primer3_code.html) (Rozen and Skaletsky, 2000) (Table 4). Loci with perfect (uninterrupted) tri-, tetra-, penta-, and hexa-repeats were chosen. Also selected were several loci with imperfect repeats, with long repeat stretches, which have the potential for replication slippage and the production of new alleles. Several imperfect repeats contain repeat stretches with different period sizes. Loci that contain invariant repeats, short repeat stretches such as (GATA)₂ (Table 2) which are not variable in a specific locus, were chosen because the repeat stretch of interest was in close proximity and primers could only be designed which included the invariant repeats. Di-nucleotide repeats were excluded because during amplification they produce more stutter bands than the larger period sizes, and are therefore more difficult to accurately score in forensics.

The 200 base pairs of flanking sequence of each locus was then compared with the total human genome sequence in GenBank, using the BLAST program, to determine if homologous sites were present elsewhere in the human genome. Primers were designed that produce products that range in size from 100-<500 bp for use in multiplex Polymerase Chain Reactions (PCR). Due to the repeated sequence in the flanking regions, primers for one locus were not designed. The resulting 62 sets of primers (Table 4) were subsequently compared with the complete genome, using the BLAST program (Altschul et al., 1990), to determine if they might amplify a product elsewhere in the genome. Several primers with multiple hits were examined manually to ensure that only one product would be produced per primer set. The primers were evaluated against themselves and with the reverse primer sequences for potential amplification products.

TABLE 2

Sixty-two loci identified from the
Y-chromosome.

| | | Reference | Reference |
|---|---|---|---|
| | Reference Allele | Allele | Allele |
| Locus | Repeat | Repeat # | Size |
| OSU57 | (CTT)₄CTTT(CTT)₃₀ | 78 | 422 |
| | (CTC)₃CTTCTC(CTT)₃ | | |
| | (CTCCTT)₄CTCCTA | | |
| | (CTT)₂₅(CTC)₂(CTT)₃ | | |
| OSU20 | (GAG)₁(AGA)₁(GAA)₃ | 61 | 358 |
| | (AGA)₁(GAG)₁(AAG)₃ | | |
| | (A)₅(GAA)₄N₁₇(AGG)₃N₆ | | |
| | (AGG)₃N₆(AGG)₃N₄ | | |
| | (AGG)₃(AAG)₁₀CAA | | |
| | (CAG)₁₁C(GGA)₁₀G(A)₅G | | |
| | (GAGAGA)₂ | | |
| OSU28 | (CTTT)₁₆(CCTT)₁ | 55 | 485 |
| | (CCTTCTTT)₅(CTTT)₂T | | |
| | (CTTT)₅T(CTTT)₃T | | |
| | (CTTT)₂T(CTTT)₁C | | |
| | (CTTT)₁₅ | | |
| OSU49 | (CTTTC)₁₂CTT(CCCT)₇T | 13 penta & | 337 |
| | (CTTTC)₁(TCTT)₅ | 41 tetra | |
| | (TCCT)₁₃(TCTT)₁₂TCT | | |
| | (TCCT)₄ | | |
| OSU21 | (AAAG)₃(A)₅(GAAA)₁₃ | 45 | 465 |
| | GAA(GGAA)₉A(GAAA)₈GA | | |
| | (GAAA)₁₂ | | |
| OSU51 | (TCTT)₁₈N₁₆(T)₆ | 40 | 388 |
| | (TCTT)₁₃TTT(TCTT)₅N₁₆ | | |
| | (T)₇ATT(ATTT)₄ | | |
| OSU55 | (TTTC)₁₅(TCTC)₂CTCC | 36 | 248 |
| (DYS449) | (TCTT)₂TCCTT(CTTT)₃ | | |
| | N₁₂(CTTT)₁₄ | | |
| OSU54 | (GAGAG)₁N₃₃(GAAA)₃N₁₉ | 15 penta & | 419 |
| | (AGAA)₁₀(AGAAG)₂AGAG | 17 tetra | |
| | (AGAAG)₁₂N₃₂(GAAG)₄ | | |
| OSU72 | (AAAGG)₄N₁₆(AGGGG)₄A | 31 | 321 |
| | (GGGAA)4AAG(AAAGG)19 | | |
| OSU64 | (AGAA)₃AGG(A)₅(AGAA)₂ | 30 | 300 |
| | (AGAG)₂AG(AGAA)₁₉(A)₃ | | |
| | GAG(A)₃(GAGA)₁(GAGG)₃ | | |
| OSU09 | (CTT)₂₃TT(CTT)₄ | 27 | 221 |
| OSU14 | (CCTT)₁₈N₇(CCCT)₃N₅ | 26 | 283 |
| | (CTCT)₂N₂₁(CTCT)₃ | | |
| OSU77 | (CCATT)₃N₉₉(ATTCC)₁₁ | 24 | 339 |
| | N₃₅(ATTCC)₁₀ | | |
| OSU59 | CTT | 22 | 451 |
| OSU46 | (GAAAG)₇GAA(GGGAA)₁₅ | 22 | 292 |
| (DYS463) | | | |
| OSU70 | (AGAGAT)₁₁N₁₀ | 22 | 388 |
| (DYS448) | (AGAGAT)₃N₁₄(AGAGAT)₈ | | |
| OSU50 | (ATAG)₂ATG(ATAG)₁₀ | 22 | 188 |
| | (ATAC)₁₀ | | |
| OSU53 | (TAC)₁₂T(ATT)₃GT | 21 | 225 |
| | (TAT)₆ | | |
| OSU52 | (GAAA)₃N₆(GAAA)₁₆ | 19 | 303 |
| (DYS458) | | | |
| OSU47 | (TCCCTT)₁₂TCCCT | 12 hexa & | 181 |
| | (CCCCT)₄C(TCCTT)₃ | 7 penta | |
| OSU31 | (TTTC)₁₇ | 17 | 201 |
| OSU35 | (AAAG)₁₇ | 17 | 432 |
| OSU76 | (AAAGG)₅N₂₆(GAAAA)₁₀ | 15 | 299 |
| OSU15 | (TCCT)₁₄ | 14 | 151 |
| OSU22 | (ATA)₁₃ | 13 | 246 |
| OSU43 | (AGAT)₁₃ | 13 | 209 |
| OSU67 | (ATT)₁₃ | 13 | 163 |
| OSU68 | (TTTTA)₁₂ | 12 | 235 |
| OSU32 | (AAAT)₁₁ | 11 | 272 |
| (DYS455) | | | |
| OSU60 | (AAAT)₁₁ | 11 | 177 |
| OSU10 | (TTAT)₁₁ | 11 | 270 |
| OSU12 | (AAAT)₁₁ | 11 | 249 |
| (DYS453) | | | |
| OSU34 | (ATA)₁₁ | 11 | 210 |
| OSU38 | (AAC)₁₁ | 11 | 381 |
| OSU40 | (ATTT)₁₁ | 11 | 252 |
| OSU56 | (AAAT)₁₁ | 11 | 247 |
| (DYS454) | | | |
| OSU66 | (AAAT)₁₁ | 11 | 146 |
| OSU42 | (ATTT)₁₁ | 11 | 348 |
| OSU11 | (AATA)₁₀ | 10 | 233 |
| OSU48 | (TGTT)₁₀ | 10 | 175 |
| OSU27 | (AAC)₁₀ | 10 | 303 |
| OSU44 | (AAAT)₁₀ | 10 | 149 |
| OSU73 | (AAT)₁₀ | 10 | 252 |
| OSU13 | (TATT)₉ | 9 | 253 |
| OSU33 | (TGT)₉ | 9 | 251 |
| OSU69 | (AATA)₉ | 9 | 346 |
| OSU74 | (CTTT)₉ | 9 | 258 |
| OSU06 | (AAACA)₈ | 8 | 416 |
| O8U16 | (TTTTG)₈ | 8 | 201 |
| OSU58 | (TTA)₈ | 8 | 293 |
| OSU62 | (GTTTT)₈ | 8 | 361 |
| OSU63 | (TATATC)₆(TATATA)₂ | 8 | 351 |
| OSU24 | (AAC)₇ | 7 | 306 |
| OSU61 | (AAAC)₇ | 7 | 204 |
| OSU65 | (TTTTG)₇ | 7 | 307 |
| OSU23 | (TTG)₇ | 7 | 300 |
| OSU37 | (AAAT)₆ | 6 | 217 |
| OSU25 | (ATTG)₅ | 5 | 350 |
| OSU71 | (AAAAC)₅ | 5 | 165 |
| OSU75 | (CCACCT)₅ | 5 | 318 |
| OSU45 | (TTTGT)₅ | 5 | 273 |
| OSU26 | (AAAAC)₄ | 4 | 203 |

Sample Collection

A test-population of 32 unrelated individuals was screened for the study: 16 Caucasian, 10 African American, 2 Hispanic and 2 East Asian males, and 2 Caucasian females. Hair and buccal samples were collected from four male individuals. Additional buccal samples were gathered from 28 individuals: 26 males and 2 females. Sixteen male buccal samples were made available by the State of Ohio Bureau of Criminal Investigation and Identification (BCI), all of which were stripped of their identifiers. The remaining 16 samples were amassed from residents of Columbus, Ohio. Each individual was provided with instructions for buccal cell collection, using sterile swabs. Participants from Columbus, Ohio, collected their own sample under supervision of the inventors. Hair samples were collected by the researcher using sterile tweezers. All tissue samples were stored at 2-8° C. until extraction.

DNA Extraction and Quantification

Samples were extracted, one at a time, at different locations in the laboratory. No extractions were conducted in the same location in one day. Three different types of DNA extractions were conducted. DNA was obtained from hair samples (follicle cells), employing a modified version of the FBI hair extraction protocol (Wilson et al., 1995). The protocol included (Austin, 1997), first, using sterile scissors to cut a 2 cm portion from the root end of the hair. The 2 cm portion was then washed in 400% of 100% ethanol in a 1.5 ml tube for 10 seconds followed by a brief rinse in 400 μl of sterile dH₂O. The hair was placed in a Kimble Kontes glass grinder (Kimble Kontes Dusseldorf, Germany) containing 100 μl of sterile TE⁻⁴. The hair was ground until all of the fragments were unable to be seen. The homogenate was transferred to a 1.5 ml plastic flip-top tube. An additional 100 μl of sterile TE⁻⁴ was added to rinse the grinder. The grinder was rinsed by pipetting up and down and the rinse was also added to the 1.5 ml tube. Microcon® concentrators 100 were replaced by Centricon® concentrators 100 (Micon® Bioseparations Millipore Corporation Bedford, Mass. formerly Micon® a GRACE company Amicon, Inc. Beverly, Mass.). Therefore, several reagent volumes were doubled. While working in a hood, 2001 of a 25:24:1 ratio of phenol:chloroform:isoamyl alcohol was added to the hair homogenate in the 1.5 ml tube. The 1.5 ml tube was vortexed on medium speed for 30 seconds then spun in a microcentrifuge for 2 minutes. From the aqueous phase of the supernatant, 180 μl was removed and placed in the Centricon®-100 which was filled with 1.5 ml of sterile TE⁻⁴ buffer. This was followed by the addition of 200 μl of sterile TE⁻⁴ to the 1.5 ml tube containing the proteinaceous interface and the organic layer. The 1.5 ml tube was again vortexed on medium speed for 30 seconds then spun in a microcentrifuge for 2 minutes. Once more 180 μl of the aqueous phase was removed and placed into the same Centricon®-100. The Centricon®-100 was covered with parafilm and a tiny hole was made with a sterile pipet tip in the center of the parafilm. The contents of the Centricon®-100 were then subjected to centrifugation at 3500 rpm for 20 minutes. The wash was removed and another 1.5 ml of sterile TE⁻⁴ was added to the same Centricon®-100. The Centricon®-100 was again covered with parafilm and a tiny hole was made with a sterile pipet tip in the center of the parafilm. The Centricon®-100 was once more subjected to centrifugation at 3500 rpm for 20 minutes. The wash was removed. An additional 100 μl of sterile TE⁻⁴ was added to the Centricon®-100. The contents of the Centricon®-100 were vortexed at medium speed. The retentate vial was added to the top of the Centricon®-100 and the Centricon®-100 was flipped over and spun in a centrifuge at 3500 rpm for 10 minutes.

DNA was obtained from buccal swabs via two different methods, either the Qlamp® DNA Mini Kit Buccal Swab Spin Protocol (QIAGEN Inc., Valencia, Calif.) or the BuccalAmp™ DNA Extraction Kit (Epicentre, Madison, Wis.) in accordance with the manufacturer's instructions. Qlamp® and hair extracted samples were stored at 2-8° C., and BuccalAmp™ extracted samples were stored at −20° C. for analysis.

DNA was also attained from buccal swabs via two different methods, either the Qlamp® DNA Mini Kit Buccal Swab Spin Protocol (QIAGEN Inc., Valencia, Calif.) or the BuccalAmp™ DNA Extraction Kit (Epicentre, Madison, Wis.) in accordance with the manufacturer's instructions. Qiamp® and hair extracted samples were stored at 2-8° C., and BuccalAmp™ extracted samples were stored at −20° C. for analysis.

The DNA was quantified, using the QuantiBlot® DNA Quantification Kit (Applied Biosystems, Foster City, Calif.) in accordance with the manufacturer's protocol. The results were visualized, using chemiluminescent detection.

PCR Amplification

The PCR conditions were optimized to facilitate multiplex reactions with previously described loci. This would allow multiplex reactions, if there is not interaction across the primer sets with previously described primer sets. Conditions developed were chosen to be compatible with previously available loci. It is not known if they interact with previously identified primer sets. The 62 loci were screened, one at a time, in uniplex reactions. Amplicons were labeled with fluorescently labeled dNTPs ([F]dNTPs). PCRs were carried out in 25-μl final volume reactions, consisting of ABI PCR Buffer II (10 mM Tris-HCL, (pH 8.3), 50 mM KCI), 2.5 mM MgCI₂, and 2.5 Units of AmpliTaq Gold (each from Applied Biosystems, Foster City, Calif.), 0.5-μM concentrations of each primer, 10 mM Bovine Serum Albumin (BSA), 200 μM of each dNTP, 0.25-0.5 μM of R110-5-UTP NEL-999 ([F]dNTP)(NEN™ Life Sciences Products Inc., Boston, Mass. 02118), and 1-3 ng of template DNA.

The PCR reactions were run in either a Perkin Elmer® Gene Amp PCR System 2400 (Perkin Elmer, Foster City, Calif.) or the Whatman Biometra® TGradient Thermocycler (Goettingen, Germany)PCR machine. The PCR conditions were as follows: 10 minute heat-soak at 95° C., 40 cycles of 1 minute at 94° C., 1 minute at 59° C., and 1 minute at 72° C., followed by a 45 minute extension time at 72° C. The following annealing temperatures for several loci were adjusted to improve amplification: OSU46 (48° C.), OSU49 and OSU50 (55° C.), OSU27 (61° C.), and OSU47, OSU72 and OSU76 (62° C.). The conditions were further optimized to remove split peaks, produced by the Taq Polymerase addition of an adenine at the end of the PCR product, by altering the final extension to 60° C. for 60 minutes.

The reactions were visualized on the ABI Prism® 310 Genetic Analyzer using GeneScan® version3.1 software (each from Applied Biosystems, Foster City, Calif.). The samples were prepared according to the manufacturer's instructions using Hi-Di™ Formamide and GeneScan® 500[ROX] size standard (Applied Biosystems, Foster City, Calif.).

Loci were named and alleles were designated according to the International Society of Forensic Genetics recommendations (Gill et al. 2001). The D#S# system will be used to name the loci and alleles were designated based on variant and non-variant repeats. Alleles were scored conservatively. One example is a tetranucleotide repeat locus which has two alleles, 234 bp and 238 bp. Any amplicon which is 232-up to but not including 236 bp was scored as 234 bp, and, subsequently, any amplicon which is 236-up to but not including 240 bp was scored as 238 bp. Therefore, variant alleles were not scored. Even in the small population tested, several loci seem to have variant alleles. Variant alleles can be determined in the future through sequencing analysis. Table 15 correlates OSU numbers to D#S# system as described above.

Multiplex

The 10 male-specific, highly variable, easy to score, and widely dispersed loci were chosen for use in two multiplex reactions. Primer sites were adjusted for use in the multiplexes. Prior to their inclusion in the multiplexes, the loci were each tested in two females to ensure that the loci are male specific. Different combinations of these loci were tested in eight males to determine the best locus combinations. Multiplex A contains five loci: OSU14, OSU35, OSU57, OSU67 and OSU77. Multiplex B is also composed of five loci: OSU9, OSU22, OSU51, OSU70, and OSU73. The PCR conditions were the same as the conditions for the uniplex reactions described above. Both multiplexes were also examined in five females to assure that no amplicons were produced due to cross-reactions between any of the five sets primer pairs.

Results

Locus Identification

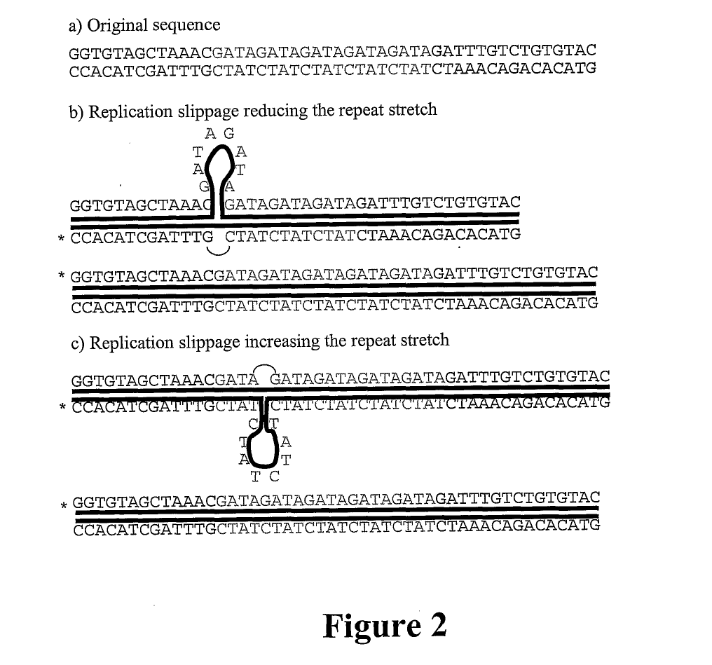

Over 17 Mb of the annotated Human Genome Sequence were screened, and 465 STR loci which are distributed across the Y-chromosome outside of the two regions containing the majority of the existing loci were identified. The period sizes of these loci are tri- to hexanucleotide repeats. The loci contain perfect repeat stretches which range in size from 4-30 repeat stretches in length. A number of loci contain more than one perfect repeat stretch, an imperfect repeat (Table 2). Of the previously available loci, several are duplicated elsewhere in the human genome. Literature searches and BLAST searches have revealed duplications on the X- and/or Y-chromosomes. The findings of Skaletsky et al. (2003), showing stretches of palindromes and inverted repeats on the Y-chromosome as well as homologous sequences on the X-chromosome, indicate that the identification of Y-STR loci unique to one location on the Y-chromosome is not a trivial pursuit. Of the 465 loci that were identified, 229 loci randomly dispersed across the Y-chromosome were examined for duplication elsewhere in the human genome by utilizing the BLAST program. The remaining 236 loci were not assessed because they are in close proximity to the clusters of loci tested. 73% of the 229 loci examined are duplicated elsewhere in the human genome, mostly on the X- and Y-chromosome (FIG. 2).

Sixty-three of the 229 loci examined by BLAST searches against the human genome were found to be unique to the Y-chromosome. The majority of the 63 loci had only one hit per primer. However, primers with multiple hits were examined manually to ensure that only one product would be produced per primer set. Each pair of forward and reverse primers was evaluated against themselves and with each other for potential amplification products. The 63 loci are dispersed across the Y-chromosome outside of the two major regions of the existing loci. Primers were unable to be created for one locus due to an extensive amount of repeats in the flanking sequence. The remaining 62 new loci include 15 trinucleotide loci, 29 tetranucleotide loci, 12 pentanucleotide loci, 3 hexanucleotide loci, 2 penta-tetranucleotide combination loci, and 1 hexa-pentanucleotide combination locus (Table 2 and Table 3). Most of the loci include only perfect repeats. However, several include imperfect repeats, which are repeats separated by insertion/deletion events or by a random sequence. Most of these repeats still have large stretches of perfect repeated sequences where replication slippage and the production of new alleles can occur. In some cases, invariant repeats were also included due to the location of the optimal primers. The products of the loci that were identified are within a size range of 100 to less than 500 bp, enabling the multiplex of several loci (Table 2 and Table 3).

TABLE 3
Number of alleles per locus in test population

| | | # of | Allelic |
|---|---|---|---|
| Locus | Repeat | Alleles | Range^{a} |
| OSU57 | tri | 12 | 393-441 |
| OSU20 | tri | 5 | 356-371 |
| OSU28 | tetra | 8 | 462-490 |
| OSU49 | penta & | 11 or 17 | 336-360 |
| | tetra | | |
| OSU21 | tetra | 11 | 446-498 |
| OSU51 | tetra | 8 | 341-409 |
| OSU55 | tetra | 9 | 237-265 |
| (DYS449) | | | |
| OSU54 | tetra & | 12 or 20 | 407-438 |
| | penta | | |
| OSU72 | penta | 1 | 322^{c}, 327 |
| OSU64 | tetra | 9 | 285-317 |
| OSU09 | tri | 9 | 213-240 |
| OSU14 | tetra | 8 | 260-292 |
| OSU77 | penta | 6 | 325-350 |
| OSU59 | tri | 9 | 449-473 |
| OSU46 | penta | 4 | 293^{c}, 273-288 |
| (DYS463) | | | |
| OSU70 | hexa | 5 | 383-407 |
| (DYS448) | | | |
| OSU50 | tetra | 5 | 185-209 |
| OSU53 | tri | 3 | 217-226 |
| OSU52 | tetra | 5 | 296-312 |
| (DYS458) | | | |
| OSU47 | penta | 4 | 172-187 |
| OSU31 | tetra | 6 | 198-210 |
| OSU35 | tetra | 7 | 425-449 |
| OSU76 | penta | 6 | 285-310 |
| OSU13 | tetra | 3 | 250-258 |
| OSU15 | tetra | 6 | 136-156 |
| OSU22 | tri | 4 | 244-256 |
| OSU43 | tetra | 5 | 202-218 |
| OSU67 | tri | 7 | 140-176 |
| OSU68 | penta | 4 | 231-246 |
| OSU32 | tetra | 5 | 261-277 |
| (DYS455) | | | |
| OSU60 | tetra | 3 | 174-182 |
| OSU10 | tetra | 3 | 267-275 |
| OSU12 | tetra | 4 | 246-258 |
| (DYS453) | | | |
| OSU34 | tri | 5 | 205-217 |
| OSU38 | tri | 3 | 376-382 |
| OSU40 | tetra | 4 | 245-257 |
| OSU56 | tetra | 3 | 244-252 |
| (DYS454) | | | |
| OSU66 | tetra | 4 | 143-155 |
| OSU42 | tetra | 4 | 341-353 |
| OSU11 | tetra | 4 | 222-238 |
| OSU48 | tetra | 3 | 172-180 |
| OSU27 | tri | 6 | 292-307 |
| OSU44 | tetra | 2 | 150-154 |
| OSU73 | tri | 6 | 250-265 |
| OSU33 | tri | 1 | 252 |
| OSU69 | tetra | 2 | 347-351 |
| OSU74 | tetra | 6 | 243-271 |
| OSU06 | penta | 3 | 417-427 |
| OSU16 | penta | 2 | 202-207 |
| OSU58 | tri | 2 | 291-294 |
| OSU62 | penta | 2 | 362-367 |
| OSU63 | hexa | 4 | 334-358 |
| OSU24 | tri | 2 | 307-310 |
| OSU61 | tetra | 2 | 201-205 |
| OSU65 | penta | 2 | 308-313 |
| OSU23 | tri | 2 | 301^{c}, 304-307 |
| OSU37 | tetra | 2 | 214-218 |
| OSU25 | tetra | 1 | 351 |
| OSU71 | penta | 2 | 166-171 |
| OSU75 | hexa | 4 | 313-331 |
| OSU45 | penta | 2 | 269-274 |
| OSU26 | penta | 1 | 204 |

^{a}Size ranges of alleles include addition of adenine by Taq Polymerase.
^{b}Compound repeats with two different repeat sizes could be scored in two ways, conservatively based upon the reference sequence by adding and subtracting four and five bases or by scoring every base pair as a new allele. The actual number of alleles is more likely closer to the upper bound than the lower bound.
^{c}Reference allele from GenBank when not observed in the 30-individual population.

According to BLAST searches and manual examinations, the 62 loci appeared to be unique to one location on the Y-chromosome. However, upon experimental examination in the test population, several primer sets produced more than one product. Nineteen loci were very difficult to score due to numerous peaks present: OSU20, OSU28, OSU72, OSU50, OSU46 (DYS463), OSU47, OSU31, OSU76, OSU13, OSU32 (DYS455), OSU34, OSU38, OSU40, OSU27, OSU69, OSU74, OSU16, OSU25 and OSU26. Other loci showed characteristics of a single duplication: OSU49, OSU21, OSU59, OSU52 (DYS458), OSU15, OSUIO, OSU42, OSU63, OSU65, OSU23, OSU37, OSU71, and OSU45. One product was observed per individual in the remaining 30 loci: OSU57, OSU51, OSU55 (DYS449), OSU54, OSU64, OSU9, OSU14, OSU77, OSU70 (DYS448), OSU53, OSU35, OSU22, OSU43, OSU67, OSU68, OSU60, OSU12 (DYS453), OSU48, OSU56 (DYS454), OSU66, OSU11, OSU44, OSU73, OSU33, OSU6, OSU58, OSU62, OSU24, OSU61 and OSU75. Even though more than one product was observed for 33 loci, new primers may be designed to obtain a single copy locus.

Variation

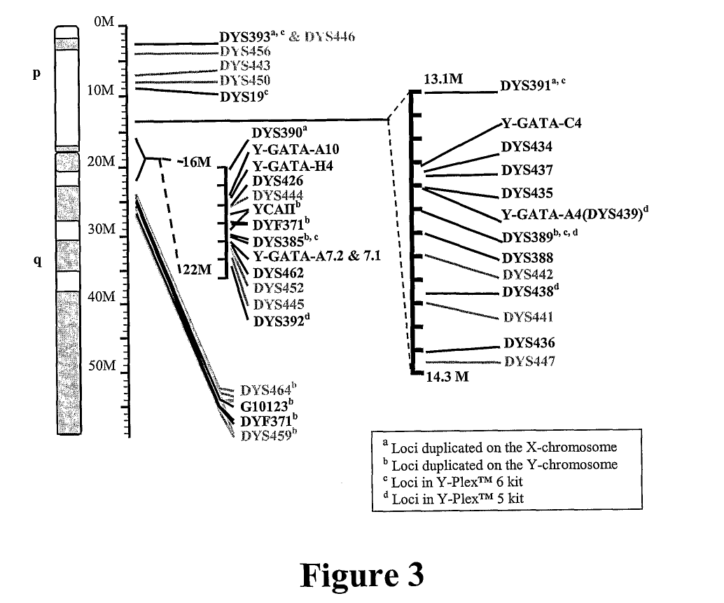

All 62 loci were screened in a small population of racially diverse individuals to assess variability. The population consisted of 16 Caucasian, 10 African American, 2 East Asian, and 2 Hispanic individuals. The schematic diagram in FIG. 3 illustrates the locations of all 62 loci on the Y-chromosome. In the 30 individuals that were screened, as many as 20 alleles per locus were found (FIG. 3 and Table 3). Forty-four percent of the 62 loci have five or more alleles (FIG. 3 and Table 3). The focus was narrowed to the 10 most appropriate loci for forensic use (OSU 10-locus set).

Criteria for the ideal loci are as follows: they should be dispersed across the Y-chromosome outside of the two concentrated regions of previously identified loci, variable between individuals, male-specific, single copy, and easy to score. Nine loci were chosen based upon the previously mentioned criteria: OSU9, OSU14, OSU35, OSU51, OSU57, OSU67, OSU70, OSU73, and OSU77 (FIG. 5). Other loci were considered but they posed several problems. For example, two tetra-pentanucleotide repeat loci, OSU49 and OSU54, although highly variable, were determined to be difficult to score. The alleles would differ by only one base pair due to the compound nature of the repeats at these loci (Table 2 and Table 3). Consequently, many amplicons would need to be sequenced to ensure a correct allele identification.

Initially, several variable loci, which were considered a part of the OSU 10-locus set, were not single copy loci, and several loci of interest were similar in size. Therefore, new primer sets were designed so that the ideal loci, based on their variability and location on the Y-chromosome, could be incorporated into a multiplex containing variable single copy loci. Loci OSU27 and OSU28 were appealing due to their location proximal to the telomeric region on the q arm. Attempts were also made to design new primers for loci OSU20 and OSU21 due to the location and variability of these loci compared to the other loci in the region. After multiple attempts to obtain single copy loci by altering the primer sites, without desirable results, OSU27, OSU28, OSU20 and OSU21 were eliminated as potential loci.

Since OSU20 and OSU21 were eliminated, OSU22 was chosen as the locus from that region. In spite of the fact that four alleles (12, 13, 14, and 16) were observed for OSU22 in the 30-individual population, it appears that in a larger population, allele 15 and additional alleles would be encountered. Once the OSU 10-locus set was determined, the discrimination power of the present loci in a 30-individual population was assessed and 30 unique haplotypes were found.

At the end of 2002, Redd et al. identified 14 new Y-STR loci. Seven of the 62 loci were also identified by Redd et al.: OSU12 (DYS453), OSU32 (DYS455), OSU46 (DYS463), OSU52 (DYS458), OSU55 (DYS449), OSU56 (DYS454), and OSU70 (DYS448) (FIG. 4). Note that the primers that were designed are not the same primer sequences designed by Redd et al. (Table 4).

TABLE 4
Primer sequences for all 62 loci

| | | | |
|---|---|---|---|
| Locus | Primer Sequence (5′-3′) | Locus | Primer Sequence (5′-3′) |

| | | | |
|---|---|---|---|
| OSU6 | F-AGCCACCTGGGTATATGAGG | OSU34 | F-GGGGTAGTGGGGAAGGATAG |
| | R-TGTTGCAGCTTTTCCTTCTG | | R-CCAGGCAATAGAGCAAGACC |
| OSU9 | F-GGCATTATGTGTTTGTGAGTGC | OSU35 | F-GAATATCCTAGCTGTGAATCTCCTC |
| | R-ACAGACTGGCAACCAAAAGG | | R-CATGGGAAAAACCCAACAC |
| OSU10 | F-AGGTTGGGTTGTGTCAACAG | OSU37 | F-CCTGGGCAACAGAGAAAGAC |
| | R-AGCAGGACTTCAGCAAGAGAG | | R-CACCACACCTGGCTAAGAAG |
| OSU11 | F-ATCCCCAAAATCTGAAATGC | OSU38 | F-TGGTGAAATCCCGTCTCTAC |
| | R-AACTGCCAGCTGAACATAAAAC | | R-TTCTTGGGGAAGGTATCAGC |
| OSU12 | F-ACCAGAAGTTAAAGGCTGTGG | OSU40 | F-AAACCACAAAAGCACATTCC |
| | R-CCTGGATGATGAACTGTAGGG | | R-ATGAGAATCGCTTGAACCTG |
| OSU13 | F-GCCAGCAGTAGACCCAGAC | OSU42 | F-AGGTGGTTTGATTTGCTTTG |
| | R-TGAGGCAGGAAAATCACTTG | | R-TCAAGAGGCTGAGGAAAGAG |
| OSU14 | F-CACCACTGTGCCAAGCTATT | OSU43 | F-TGATGGATAGAAACACAGAAATACA |
| | R-CAGAGCAACCCTCTGTCAAG | | R-TTACAACCCTGCAAAGGAAG |
| OSU15 | F-TGGGAAACTGATCCAAACC | O8U44 | F-AGGCAGAGGTTCCAGTAAGC |
| | R-GGGTTACTTCGCCAGAAGG | | R-GGATGCTGGGTCAAACAGTAG |
| OSU16 | F-AAACCATCCTTGCATCACAG | OSU45 | F-AGAACTTTGGCAGACTTTGTG |
| | R-CCAAAACCAGACAAACACCTC | | R-AGGTGGGAGGATTGTTTGAG |
| OSU20 | F-AATGGAGATTGGACATGCTG | OSU46 | F-TGAGAAAAGTCTCGCCTTACC |
| | R-CAGTTGAAGGTAAAGCAAAATCC | | R-GAGGCATGAGGTTGTGTGAC |
| O8U21 | F-GTGACTGGAGAACTGCTGGA | OSU47 | F-CCTAAAAGTTACAACCCAGCAC |
| | R-TTCCTTTTGGTTTTATGCCTTT | | R-GCCTGGTGACAGAGTGAGAC |
| OSU22 | F-TTGTGCTCATGTACCCTGGA | OSU48 | F-GAGGGGAGTGTAGAAAGAATGC |
| | R-CCTCCTGTCTGCCATTTTGT | | R-AGGGGGCTGAGTAATGGAG |
| OSU23 | F-GTTGTCCGGCTTTTTGAGTT | OSU49 | F-CCAAATAAACTGTGGATGGAAG |
| | R-CTCCCACAGGAAGAAGAAGG | | R-GCAACAGGGGGAATACTCTG |
| OSU24 | F-TTGCTTGTACCCAGAAGACG | OSU50 | F-CTGCCCAACATAGTGAAACC |
| | R-AGGAATTGGACCCCTCAATC | | R-GAGATTACAGGCACCACCATC |
| OSU25 | F-TTGCAGTAAGCGGAGATCG | OSU51 | F-CTGGGTGTGCATTCGAGAC |
| | R-AAATGGAAAGCAAACCTTGG | | R-CCTGGGTGACAGACTCCATC |
| OSU26 | F-TGAGGCAGGAGAATAGCTTG | OSU52 | F-GCTGCCTCTAATGTGAGCTG |
| | R-TGAGAGACTTCCCACTCCAC | | R-AGGATGGTCTCGATTTCCTG |
| OSU27 | F-GGAAGGGGAACATCACACTC | OSU53 | F-ACTGTCACCCCTTGACTGAG |
| | R-ACGGTCTCAATCTCCTGACC | | R-GAAGCTGAGGCAGGAGAAAG |
| OSU28 | F-CTGTTCTGCTGTTGGCTGAC | OSU54 | F-ACTTGGGTGGGTGTTACTGG |
| | R-ACATGGTAAAACCCCGTCTC | | R-TTGAGGATAATGGGCAAAATG |
| OSU31 | F-GAAATCCTGGCTGTGTCCTC | OSU55 | F-TTTTTCTTGCTCTTTTTCTTTTCTC |
| | R-TCTAAGGGATGCAAGGTGTG | | R-TTGCACCATTGCACTCTAGG |
| OSU32 | F-CTAAGCCCACAAGGTCAAGG | OSU56 | F-GCAGTAGGAAGGCTGGAGAC |
| | R-CATTCAGCAGCCAGTGATTC | | R-TTCTTTGGCCCTGCATTTAC |
| OSU33 | F-AGAGTGCCCTTGTATTGCAG | OSU57 | F-GAAATTGTGACATACCGCTGAC |
| | R-CTGAGGCAGGAGAATTGTTG | | R-CGAGCAACAGTGCAAGACTC |
| OSU58 | F-CATGTTACCCACCTCTCCTG | OSU68 | F-TGGCTGTACTCTATTCCAGGTTC |
| | R-GCAGCACTCCAAAATGACAG | | R-TGACGAGTTAGTGGGTGCAG |
| OSU59 | F-GGGTTGCTTTCTGCTAGGTG | OSU69 | F-CATGCACCTGTAATCCCAAC |
| | R-TGGTGTGCTTCTCTTCCTTC | | R-CTTCACCCTCAAAAGCAATG |
| OSU60 | F-CTGGCATTCAAATCCTCTCC | OSU70 | F-GGTGGGTTTTAGTTGGCTATG |
| | R-CAGTGTCTCTTCCTGGGTTG | | R-TTCTTGATTCCCTGTGTTGG |
| OSU61 | F-AAAGAAGAGAAGCACACCACAC | OSU71 | F-TTTTTCTGTGGGTCTGAATCC |
| | R-GTCCCAATATGATGGAAGAGG | | R-CCTGGGAGATGTCTGTTTTTC |
| OSU62 | F-CTCCCACAGAAACACACACC | OSU72 | F-AGAGTCTAGGGCGACAGAGC |
| | R-ACCCAGTGAAAACCCATCTC | | R-TGCCATTTAGATTGTGGTTTG |
| OSU63 | F-GAAGTGCGTGTCCTCACCTA | OSU73 | F-TGCTTGAACCTTGGAGACAG |
| | R-TTTGTTTCCCTCTCCTTCTCA | | R-TTGACTTGTTGACCCTGTGG |
| OSU64 | F-AGCACAGATAATGCCACTGC | OSU74 | F-GCTGAGATTACTGGTGTGAGC |
| | R-TCTCCTTCGTTCCTTCCTTC | | R-CATGTTGCTGGGAGTGAGAC |
| OSU65 | F-GGCAAGATGAACAAGGTGTC | OSU75 | F-CTCTCCAGCTTTTCCCACTG |
| | R-ACTGGAGGGAACCAACTCTG | | R-GGGCACCATTTTCAGGATAG |
| OSU66 | F-ATTGGGTGACAACACTCCAG | OSU76 | F-GGTTGAGGTGGGAGAATAGC |
| | R-GTAAGCGTGGGAAAACAATG | | R-GGCCCAGTAGCAATACAGTG |
| OSU67 | F-TCAGGAGAAAATTCCAAAAGC | OSU77 | F-ATTATATCCCGTCCGATTCC |
| | R-CAGTGAGCCAAGATGGTGAC | | R-TTGGTGTGAACTGGAGTGG |

Since these loci were already examined in the sample population, a direct comparison of the average number of alleles for the seven Redd loci with the average number of alleles for the OSU 10-locus set was possible. It was determined that in the same 30 individuals, the OSU 10-locus set had an average of 2.5 more alleles per locus than the seven Redd loci (Table 5). Note that one locus OSU70 (DYS448) is the same for both sets.

TABLE 5
Average number of alleles per locus for seven Redd loci
and OSU 10-locus set in the same 30 individuals.

| OSU 10-Locus | Number of | | Number of |
|---|---|---|---|
| Set | Alleles | Redd Loci | Alleles |
| OSU9 | 9 | OSU12 | 4 |
| | | (DYS453) | |
| OSU14 | 8 | OSU32 | 5 |
| | | (DYS455) | |
| OSU22 | 4 | OSU46 | 3 |
| | | (DYS463) | |
| OSU35 | 7 | OSU52 | 5 |
| | | (DYS458) | |
| OSU51 | 8 | OSU55 | 9 |
| | | (DYS449) | |
| OSU57 | 12 | OSU56 | 3 |
| | | (DYS454) | |
| OSU67 | 9 | OSU70 | 5 |
| | | (DYS448) | |
| OSU70 | 5 | | |
| (DYS 448) | | | |
| OSU73 | 6 | | |
| OSU77 | 6 | | |
| Average | 7.4 | Average | 4.9 |
| | Difference | 2.5 | |

Multiplex

Two multiplex reactions were designed to screen a larger population more effectively. As previously stated, several primer sites were adjusted to produce single copy loci and for incorporation into a multiplex. Each multiplex contains five loci. The loci were grouped together based upon trial and error to obtain loci that work best together in a single amplification. Multiplex A consists of OSU14, OSU35, OSU57, OSU67, and OSU77. Multiplex B consists of OSU9, OSU22, OSU51, OSU73, and OSU77. The two multiplexes were tested in five females to ensure that there was no cross-reactivity between primer sets for sites outside the Y-chromosome. The final primer sequences for all 10 loci are listed in Table 4 along with the original primer sequences for the remaining 52 loci.

Allelic Distribution of the OSU 10-Locus Set

However, some differences may exist in the allelic distributions for as many as nine of the loci were observed when compared to the African American and Caucasian populations. The East Asian and Hispanic individuals were not considered in this assessment because they are each represented by only two individuals.

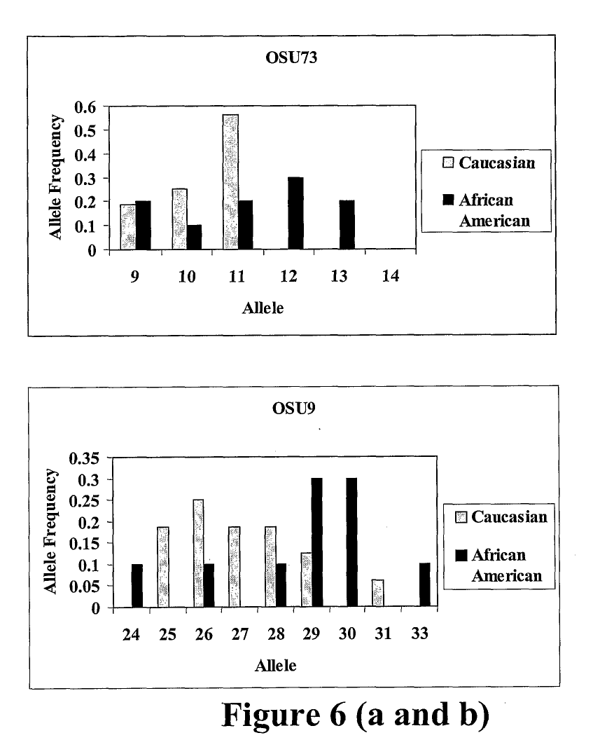

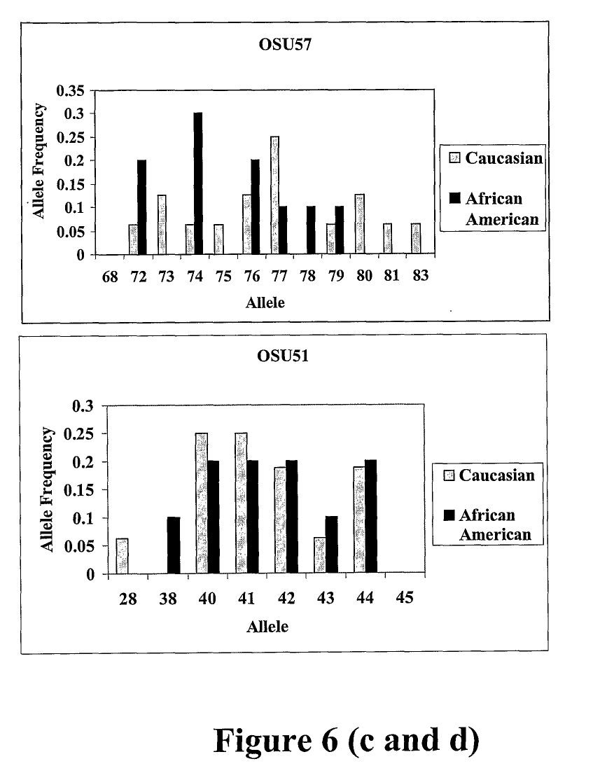

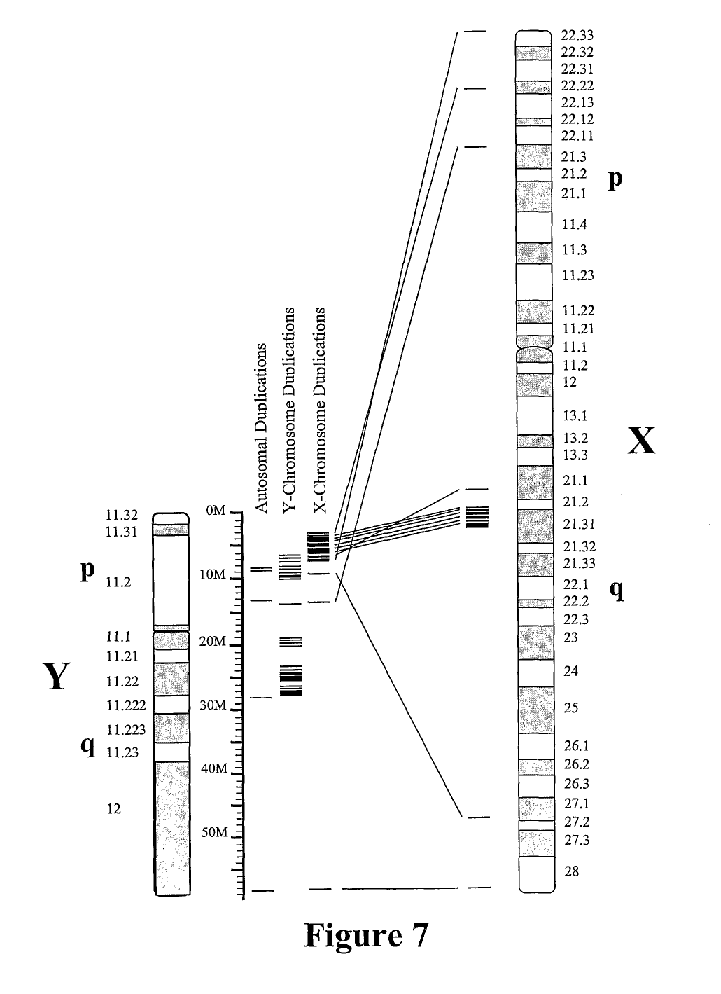

FIG. 6 depicts four loci from the present 10-locus set: three examples of loci with different allelic distributions and the only locus out of all 10 with little disparity in the allelic distribution for the Caucasian and African American populations. OSU73 (FIG. 6a) displayed a different allelic distribution for the two populations. In the 30-individual population, six alleles were observed for OSU73. The most common allele in the Caucasian population is 11 whereas 12 is the most common allele for the African American population. Also observed were a different allelic distribution for both populations with OSU9 (FIG. 6b). At this locus, 9 alleles were observed for the whole population. The 29 and 30 alleles were the most common in the African American population while the 26 allele was the most common in the Caucasian population. Additionally, OSU57 (FIG. 6c) exhibited a different allelic distribution for each population. A total of 12 alleles were detected in the test population. The modal allele for the Caucasian and African American populations is 77 and 74, respectively. There was no apparent allelic distribution for OSU51 (FIG. 6d), which distinguished the Caucasian and African American populations. A total of eight alleles were identified in the population. Four alleles 40, 41, 42, and 44 were nearly equivalent and are the most common alleles for both populations.

At this time, due to the small population sample sizes, it is unclear whether ethnic specific allelic associations occur for any locus. Also, it should be noted that the haplotypes that were observed did not seem to segregate Caucasians from African Americans. A more extensive survey (more individuals-male and female) is to be performed on all loci.

Discussion

New Y-STRs

Y-STRs are powerful tools. They can be used in the identification of degraded or limited male samples, particularly in female/male body fluid mixtures, and the identification of the number of rapists in a multiple rape. However, the use of markers that exist at multiple Y-chromosome locations defeats this purpose, particularly with degraded samples. Y-STR primer sets that also generate amplification products from the X-chromosome are no more useful in male/female mixed samples than autosomal STRs. STR primer sequences that amplify multiple loci on the Y-chromosome are also problematic.

According to Redd et al. (2002), the multiple copy loci were the most variable group of loci that have been identified. Based upon the number of alleles that have been observed in a small population, in contrast to the number of alleles reported in the literature for the previously identified loci, the single copy loci reported here rival the results for multicopy amplification. Moreover, there are several problems seen with the use of multiple copy loci. For example, one to three alleles have been observed for DYS385, and one to four alleles have been observed for DYS464. When single individuals are studied, it is difficult to accurately score multicopy loci in forensic samples, which may be limited and/or degraded because of the uncertainty of the number of alleles in any individual. Additionally, if duplicated loci are the only variable loci used and allelic dropout is a potential problem in degraded forensic samples with multicopy loci, the discrimination power of the set of loci examined is significantly reduced. Allelic dropout may cause the incorrect exclusion of a suspect. This is true even more so than with an autosomal locus since the “allele” frequencies are not independent and cannot be multiplied due to the assumption of no recombination and complete linkage.

The use of single copy loci eliminates many problems associated with multiple copy loci. This is particularly true for samples that contain multiple male individuals, in which the concentration of individual contributions is unknown.

Highly variable single copy STRs are easier to score than duplicated loci, and are discriminative. The most important criteria for a forensic Y-STR marker is that they are male-specific, variable, and easily scored. The single copy loci that fit the aforementioned criteria were identified. Additionally, several loci identified here may be more variable than shown in these studies. Alleles were scored conservatively in this study. Based upon the gene scan values seen in the electropherograms, there is evidence for the presence of some variant alleles. Sequencing analysis of these alleles must be completed to confirm their existence.

The work that has been done exhibits the potential of the loci. In a subsequent study, a comparative analysis of the OSU 10-locus set was conducted with the 10 most widely used Y-STR loci on the same population of 30 individuals (Example 2).

Electronic-Database Information

The URLS for databases and software mentioned in this article are as follows: European Y-STR database, http://www.ystr.org/europe; USA Y-STR database, http://www.ystr.org/usa; National Center for Biotechnology Information (NCBI), http://www.ncbi.nlm.nih.gov; and GDB, http://www.gdb.org.

### Example 2

### Comparison of 10-Locus Set with Commercially Available Sets

Direct comparisons were made between the OSU 10-locus set and the 10 Y-STR markers present in the Reliagene Y-PLEX™ 6 and Y-PLEX™ 5 kits to evaluate the discrimination power for each set in the 30-individual test population.

Materials and Methods

Polymerase Chain Reactions (PCRs)

The 10 OSU loci were screened one at a time in uniplex reactions. Amplicons were labeled with fluorescently labeled dNTPs ([F]dNTPs). PCRs were carried out in 25 μl final volume reactions, consisting of ABI PCR Buffer 11 (10 mM Tris-HCL, (pH 8.3), 50 mM KCl), 2.5 mM MgCl₂, and 2.5 Units of AmpliTaq Gold (each from Applied Biosystems, Foster City, Calif.), 0.5-μM concentrations of each primer, 10 mM Bovine Serum Albumin (BSA), 200 μM of each dNTP, 0.25-0.5 μM of R110-5-dUTP NEL-999 ([F]dNTP) (NEN™ Life Sciences Products Inc., Boston, Mass. 02118), and 1-3 ng of template DNA. The PCR reactions were run in either a Perkin Elmer® Gene Amp PCR System 2400 (Perkin Elmer, Foster City, Calif.) or Whatman Biometra® TGradient Thermocycler (Goettingen, Germany) PCR machine. The PCR conditions were as follows: 10-minute heat-soak at 95° C., 40 cycles of 1 minute at 94° C., 1 minute at 59° C., and 1 minute at 72° C., followed by a 45 minute extension time at 72° C. The conditions were further optimized to remove split peaks, produced by the Taq Polymerase addition of an adenine at the end of the PCR product, by altering the final extension to 60° C. for 60 minutes.

PCR conditions for the Y-PLEX kits were performed, following the manufacturer's instructions (Reliagene, New Orleans, La.). The reactions were visualized on an ABI Prism® 310 Genetic Analyzer, using GeneScan® version 3.1 software (each from Applied Biosystems, Foster City, Calif.). The OSU 10-locus set samples were prepared according to Applied Biosystems' instructions for visualization of PCR using the 310 Genetic Analyzer, using Hi-Di™ Formamide and GeneScan®-500 [ROX] size standard (Applied Biosystems, Foster City, Calif.). The Y-PLEX samples were also prepared, according to the manufacturer's instructions (Reliagene, New Orleans, La.). Genotyper® software (Applied Biosystems, Foster City, Calif.) was used to score the alleles of the Y-PLEX loci, utilizing the allelic ladders provided with both kits (Reliagene, New Orleans, La.).

Genetic Analysis

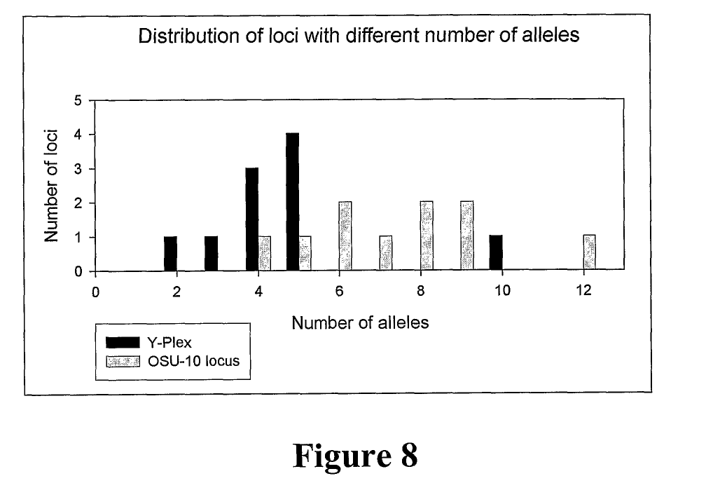

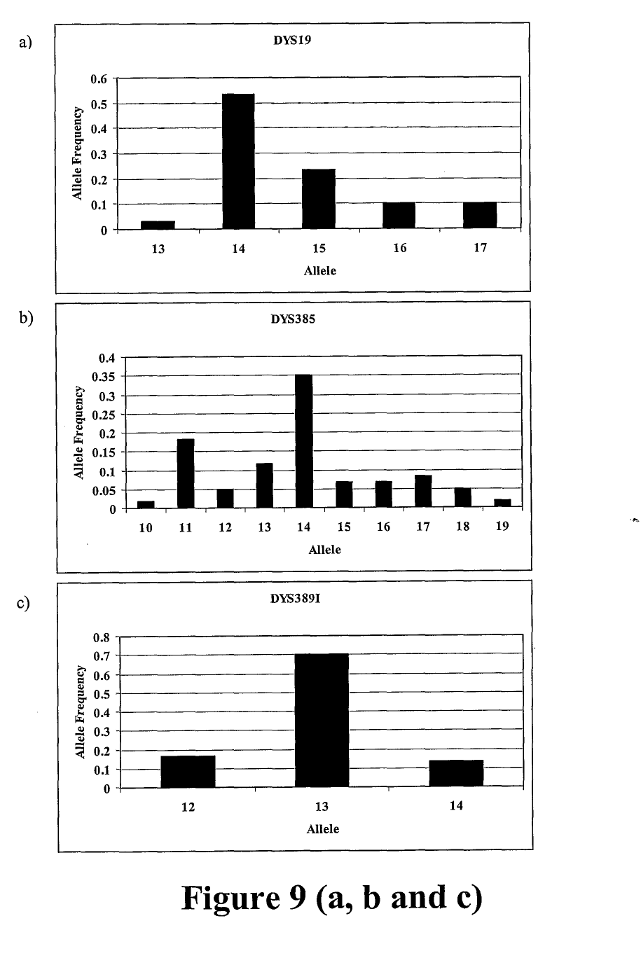

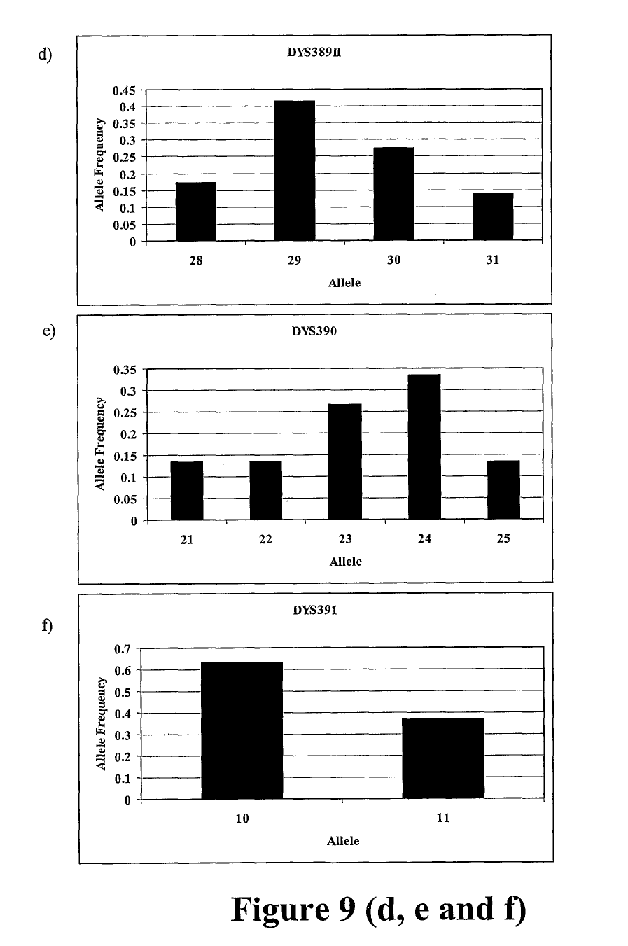

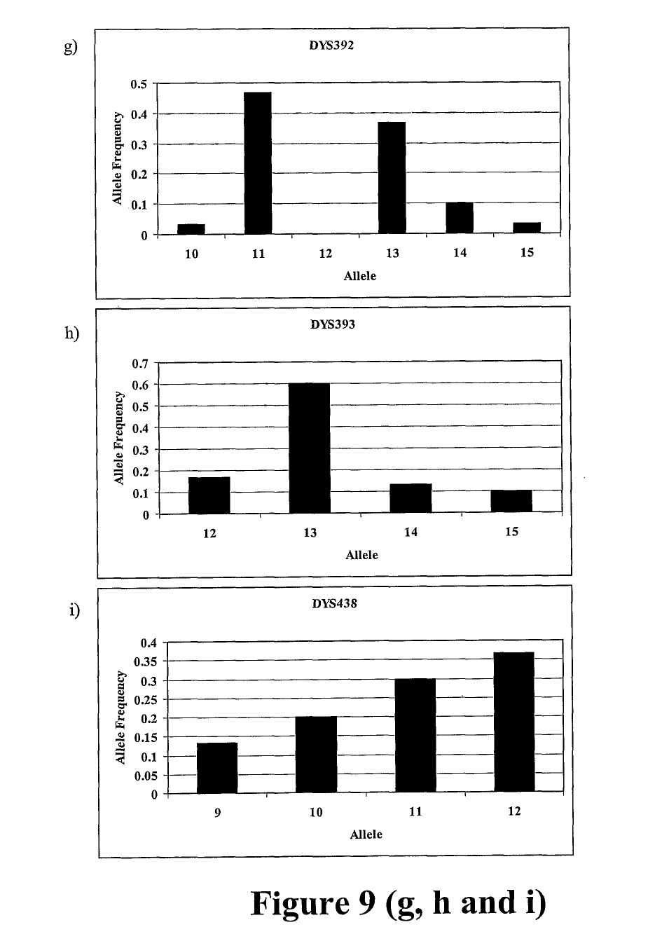

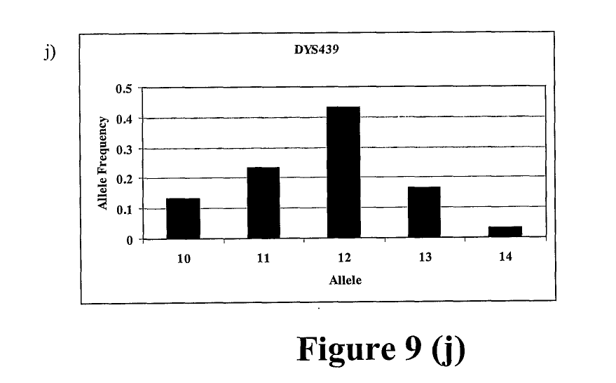

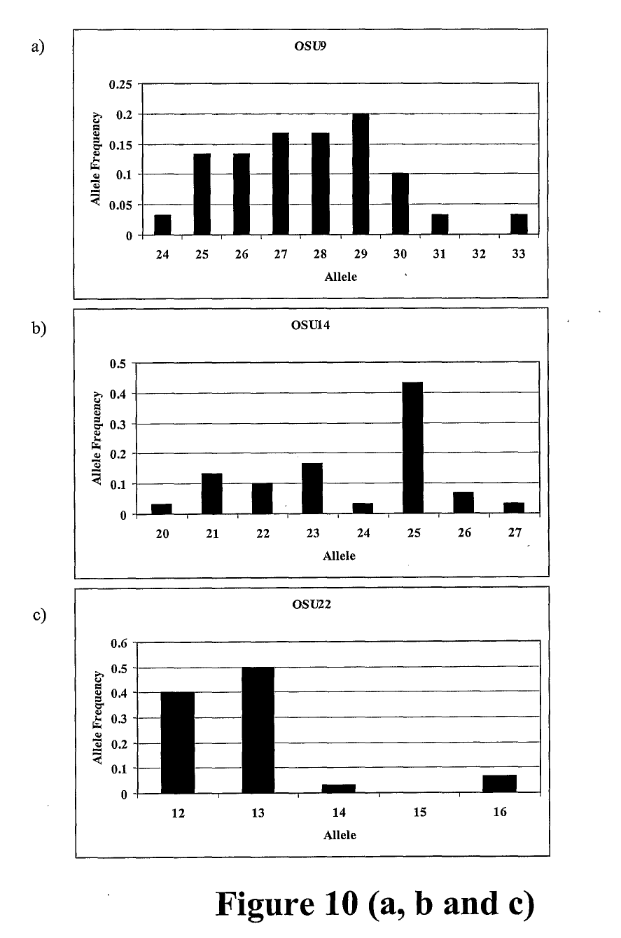

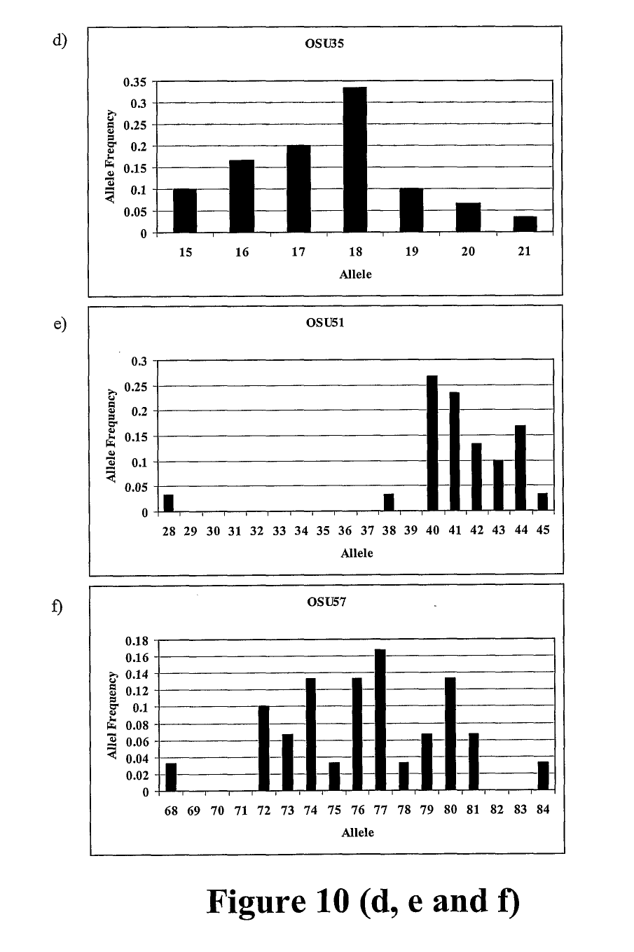

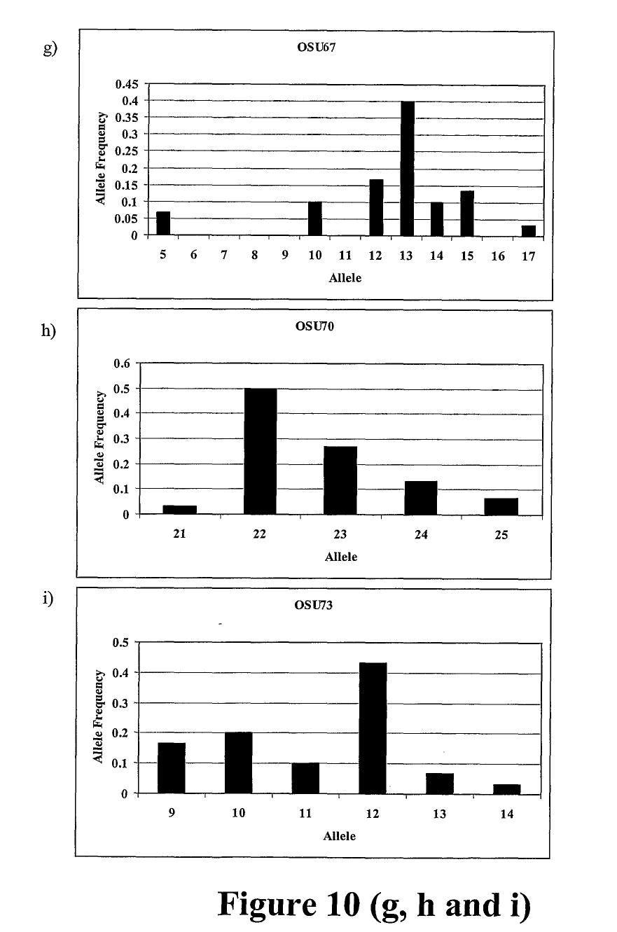

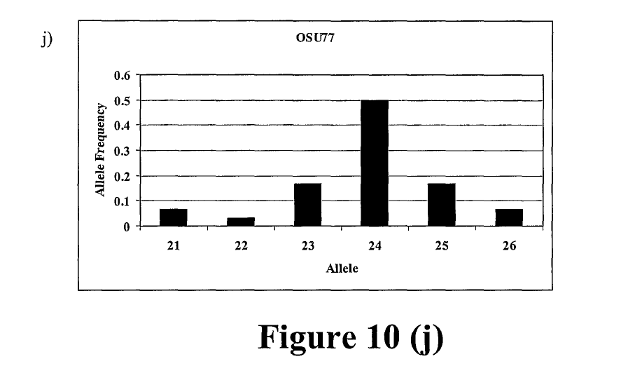

The number of alleles observed in the 30-individual test population for all 20 loci were evaluated (FIG. 8). Allele frequencies (Table 6 and 7 and FIG. 9 and 10), gene diversities (Table 8) and independent segregation analyses (Tables 9 to 14) were calculated using Genepop on the Web software v.3.4 Option5 and Option2 (Raymond and Rousset 1995) for both sets of loci. The p-values for the linkage disequilibrium analyses were calculated, using Fisher's exact test. To calculate significance, the independent segregation analysis utilized a Markov Process to resample the data with the following parameters: a dememorization of 1000, 1000 batches, and 5000 iterations per batch. Analysis of independent segregation among pairs of loci was conducted for the population as a whole, and, separately, for the African American and Caucasian subgroups. When pairs of loci are compared, there are 45 pairwise tests each between loci within the OSU 10-locus set and between loci within the Y-PLEX set of loci for each population group. In addition, when comparisons are made between loci, one from each of the two sets, 100 additional pairwise comparisons of independent segregation can be obtained for each population group or subgroup.

[symbol]

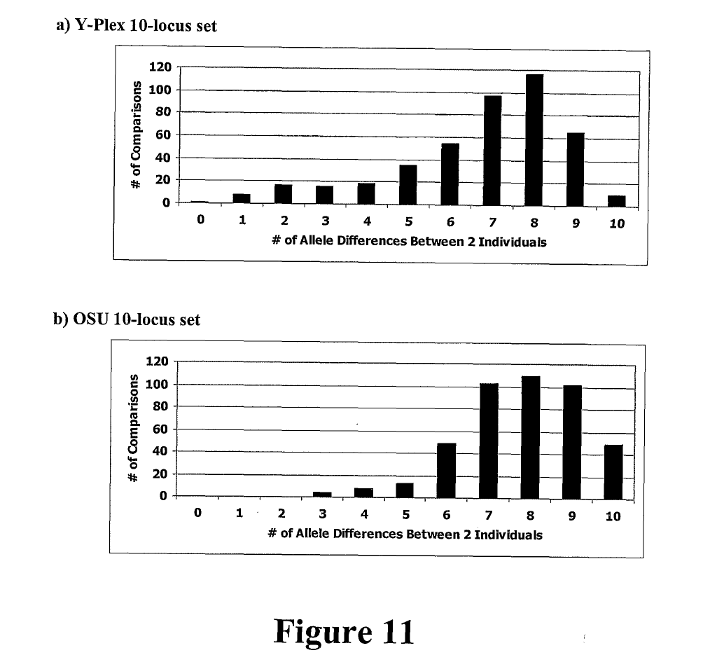

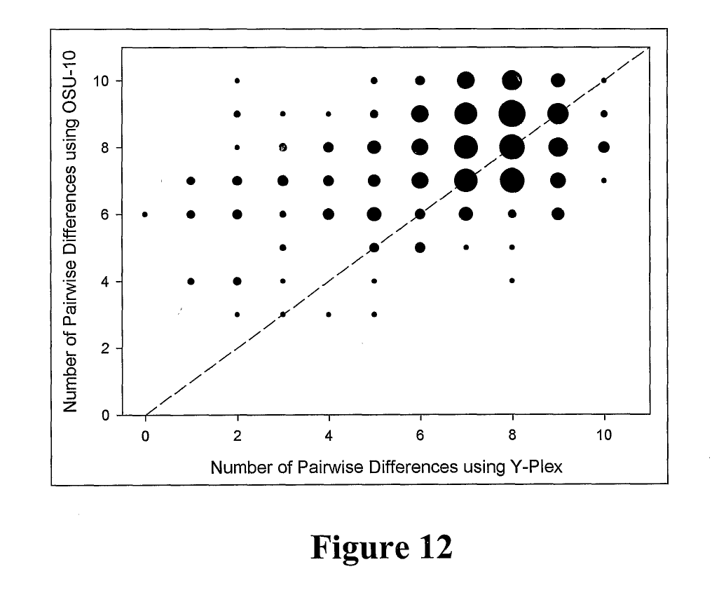

The discrimination power of both sets of 10 loci was evaluated by conducting side-by-side examinations with the same 30 individuals. The first test involved a comparison of the number of haplotypes for both sets of loci. A pairwise comparison was then conducted by examining every individual with every other individual and noting the number of differences between each pair for each set of loci (FIG. 11). In order to directly compare the discrimination power of the two sets of loci, data were plotted for the two sets with every pair (FIG. 12).

Results

Allelic Comparisons

Based upon an initial screen of the 30-individual test population, the OSU 10-locus set appears to be more informative than other sets of loci. The OSU 10-locus set revealed 30 unique haplotypes in the 30-individual population. During the screen for new loci, described in Example 1, seven of 62 loci were common with Redd et al. (2002). When the average number of alleles per locus was compared with the aforementioned seven locus panel in the 30-individual sample population, it was found that the OSU 10-locus set had an average of 2.5 more alleles per locus than the seven Redd loci. Note that one locus occurs in common between the two sets.

To further examine the discriminative power of the OSU 10-locus set, a comparative study was conducted against the set of 10 loci that are contained in the Y-PLEX kits, produced by Reliagene, which are widely used in forensics and other population analyses (loci shown in FIG. 3). The number of alleles for all 20 loci examined in the same 30 individuals was compared in FIG. 8. The Y-PLEX loci represented by black bars contained an average of 4.7 alleles per locus. For the nine single copy loci, two to five alleles were observed, and, for the multicopy locus, DYS385, 10 alleles were observed. The OSU loci represented by gray bars showed an average of 7.4 alleles per locus. All 10 OSU loci are single copy, and from four to 12 alleles were observed. Therefore, in the same 30 individuals, an average of 2.7 more alleles per locus were observed, using the OSU 10-locus set.

The allele frequencies for the Y-PLEX set and the OSU 10-locus set are presented in Tables 6 and 7 and are represented graphically in FIGS. 9 and 10, respectively. With the exception of DYS392 and DYS385, all of the Y-PLEX loci show a unimodal distribution (FIG. 9). In contrast with the Y-PLEX loci, five OSU loci have a unimodal distribution (FIG. 10). At several loci, alleles were absent. In the 30-individual test population, the following was observed: nine alleles for OSU9, OSU24 to OSU31 and OSU33 (Table 7 and FIG. 10a), four alleles for OSU22, OSU12 to OSU14 and OSU16 (Table 7 and FIG. 10c), nine alleles for OSU51, OSU28 and OSU38 to OSU45 (Table 7 and FIG. 10e), 12 alleles for OSU57, OSU68, OSU72 to OSU81, and OSU84 (Table 7 and FIG. 10f), seven alleles for OSU67; the range is interrupted three times, 5, 10, 12 to 15, and 17 (Table 7 and FIG. 10g), five alleles for DYS392, DYSIO to DYS11, and DYS13 to DYS15 (Table 6 and FIG. 9g).

TABLE 7
OSU 10-locus set allele frequencies

| | Locus | | | | | | | | | |
|---|---|---|---|---|---|---|---|---|---|---|
| Allele | OSU09 | OSU14 | OSU22 | OSU35 | OSU51 | OSU57 | OSU67 | OSU70 | OSU73 | OSU77 |
| 5 | | | | | | | 0.067 | | | |
| 9 | | | | | | | | | 0.167 | |
| 10 | | | | | | | 0.1 | | 0.2 | |
| 11 | | | | | | | | | 0.1 | |
| 12 | | | 0.4 | | | | 0.167 | | 0.433 | |
| 13 | | | 0.5 | | | | 0.4 | | 0.067 | |
| 14 | | | 0.033 | | | | 0.1 | | 0.033 | |
| 15 | | | | 0.1 | | | 0.133 | | | |
| 16 | | | 0.067 | 0.167 | | | | | | |
| 17 | | | | 0.2 | | | 0.033 | | | |
| 18 | | | | 0.333 | | | | | | |
| 19 | | | | 0.1 | | | | | | |
| 20 | | 0.033 | | 0.067 | | | | | | |
| 21 | | 0.133 | | 0.033 | | | | 0.033 | | 0.067 |
| 22 | | 0.1 | | | | | | 0.5 | | 0.033 |
| 23 | | 0.167 | | | | | | 0.267 | | 0.167 |
| 24 | 0.033 | 0.033 | | | | | | 0.133 | | 0.5 |
| 25 | 0.133 | 0.433 | | | | | | 0.067 | | 0.167 |
| 26 | 0.133 | 0.067 | | | | | | | | 0.067 |
| 27 | 0.167 | 0.033 | | | | | | | | |
| 28 | 0.167 | | | | 0.033 | | | | | |
| 29 | 0.2 | | | | | | | | | |
| 30 | 0.1 | | | | | | | | | |
| 31 | 0.033 | | | | | | | | | |
| 33 | 0.033 | | | | | | | | | |
| 38 | | | | | 0.033 | | | | | |
| 40 | | | | | 0.267 | | | | | |
| 41 | | | | | 0.233 | | | | | |
| 42 | | | | | 0.133 | | | | | |
| 43 | | | | | 0.1 | | | | | |
| 44 | | | | | 0.167 | | | | | |
| 45 | | | | | 0.033 | | | | | |
| 68 | | | | | | 0.033 | | | | |
| 72 | | | | | | 0.1 | | | | |
| 73 | | | | | | 0.067 | | | | |
| 74 | | | | | | 0.133 | | | | |
| 75 | | | | | | 0.033 | | | | |
| 76 | | | | | | 0.133 | | | | |
| 77 | | | | | | 0.167 | | | | |
| 78 | | | | | | 0.033 | | | | |
| 79 | | | | | | 0.067 | | | | |
| 80 | | | | | | 0.133 | | | | |
| 81 | | | | | | 0.067 | | | | |
| 84 | | | | | | 0.033 | | | | |

The gene diversity was calculated for every locus (Table 8). DYS385 was evaluated as two different loci. The gene diversity for the Y-PLEX 10-locus set ranged from 0.472 to 0.807. The gene diversity for the OSU 10-locus set was from 0.594 to 0.906. The average gene diversity was 10% higher in the OSU 10-locus set. Four loci in the OSU 10-locus set had higher gene diversities than the most diverse locus, DYS385a, in the Y-PLEX set.

TABLE 8
Gene diversity for OSU and Y-PLEX 10-locus sets.

| | |
|---|---|
| Locus | Gene Diversity |

OSU

| | |
|---|---|
| OSU57 | 0.906 |
| OSU9 | 0.870 |
| OSU51 | 0.829 |
| OSU35 | 0.809 |
| OSU67 | 0.782 |
| OSU14 | 0.762 |
| OSU73 | 0.741 |
| OSU77 | 0.696 |
| OSU70 | 0.667 |
| OSU22 | 0.594 |
| Average | 0.766 |

Y-PLEX

| | |
|---|---|
| DYS385a | 0.807 |
| DYS390 | 0.777 |
| DYS438 | 0.730 |
| DYS439 | 0.723 |
| DYS389II | 0.692 |
| DYS19 | 0.651 |
| DYS392 | 0.646 |
| DYS385b | 0.644 |
| DYS393 | 0.594 |
| DYS389I | 0.472 |
| DYS391 | 0.472 |
| Average | 0.655 |

Haplotype Comparisons

A comparative analysis of haplotypes was conducted between the Y-PLEX and OSU 10-locus sets, since these sets have an equal number of loci. Each of the 30 individuals of the sample population was compared with every other individual, in a pairwise fashion, to determine the number of differences between each pair of individuals (FIG. 10) for each set of loci. The OSU 10-locus set shows an average of one additional difference between individuals (7.79 versus 6.78 differences per comparison) compared to the Y-PLEX loci. The distribution of pairwise differences is shown in FIGS. 11a and 11b for the two sets of loci. For the Y-PLEX 10-locus set, 40 pairs of individuals have 0-3 differences (FIG. 11a), whereas only four pairs of individuals differ at three loci, using the OSU set, and none show less than three differences (FIG. 11b). All 30 haplotypes are unique for the OSU 10-locus set while one pair of individuals shares the same haplotype, using the Y-PLEX kits (FIG. 11a and 11b). This same pair of individuals differs by six loci with the OSU 10-locus set (FIG. 12). Additionally, twice as many pairs differ by nine or 10 loci with the OSU 10-locus set when compared with the Y-PLEX 10-locus set (FIG. 11).

The comparison of the OSU 10-locus set and the loci of the Y-PLEX sets is further shown in FIG. 11. This figure displays a comparison of the number of differences observed between specific pairs of individuals, utilizing the OSU-10-locus set and the Y-PLEX set. The data show a skew toward a greater number of differences observed with the OSU 10-locus set (points above the diagonal).

Linkage Disequilibrium Comparisons

Linkage disequilibrium was calculated for the population as a whole as well as separately for the African American and Caucasian populations for both sets of loci (Table 9, Table 10, Table 11, Table 12, Table 13, and Table 14). In the 30-individual population, more linkage disequilibrium was observed with the Y-PLEX set (Table 9) than with the OSU set (Table 12) of loci. Examination of the Y-PLEX set at a P-value of less than 0.01 showed 12 pairs of loci in linkage disequilibrium and at a P-value of less than 0.05 revealed 19 pairs of loci in linkage disequilibrium. DYS438 was in linkage disequilibrium with nearly every locus in the Y-PLEX set. Nine of the 10 Y-PLEX loci were in linkage disequilibrium with at least one locus at a P-value of less than 0.01. All 10 Y-PLEX loci were in linkage disequilibrium with at least one locus at a P-value of less than 0.05.

TABLE 9
Linkage disequilibrium analysis of
Y-PLEX loci in all 30 individuals

| | | | | Standard |
|---|---|---|---|---|
| Locus 1 | Locus 2 | Chi2 | P-Value | Error |
| DYS390 | DYS438 | Infinity | Highly Significant | 0 |
| DYS385 | DYS438 | Infinity | Highly Significant | 0 |
| DYS438 | DYS392 | Infinity | Highly Significant | 0 |
| DYS391 | DYS392 | 23.026 | 0 | 0 |
| DYS391 | DYS438 | 18.631 | 0 | 0 |
| DYS389I | DYS438 | 13.756 | 0.001 | 0 |
| DYS390 | DYS389I | 13.434 | 0.001 | 0 |
| DYS390 | DYS392 | 13.23 | 0.001 | 0 |
| DYS389II | DYS389I | 12.239 | 0.002 | 0 |
| DYS19 | DYS390 | 12.39 | 0.002 | 0.001 |
| DYS393 | DYS385 | 11.632 | 0.003 | 0.001 |
| DYS393 | DYS438 | 9.622 | 0.008 | 0.001 |
| DYS19 | DYS439 | 8.675 | 0.013 | 0.002 |
| DYS390 | DYS439 | 8.162 | 0.017 | 0.002 |
| DYS19 | DYS392 | 7.733 | 0.021 | 0.003 |
| DYS385 | DYS389I | 7.186 | 0.028 | 0.005 |
| DYS439 | DYS438 | 6.736 | 0.034 | 0.003 |
| DYS393 | DYS390 | 6.72 | 0.035 | 0.003 |
| DYS390 | DYS385 | 6.612 | 0.037 | 0.007 |
| DYS385 | DYS392 | 5.874 | 0.053 | 0.010 |
| DYS390 | DYS391 | 5.485 | 0.064 | 0.002 |
| DYS391 | DYS385 | 5.335 | 0.069 | 0.005 |
| DYS389II | DYS439 | 4.776 | 0.092 | 0.005 |
| DYS389I | DYS392 | 4.674 | 0.097 | 0.005 |
| DYS389II | DYS438 | 4.403 | 0.111 | 0.005 |
| DYS393 | DYS389I | 4.305 | 0.116 | 0.005 |
| DYS391 | DYS389I | 4.288 | 0.117 | 0.003 |
| DYS19 | DYS389I | 4.158 | 0.125 | 0.005 |
| DYS393 | DYS389II | 4.062 | 0.131 | 0.006 |
| DYS389II | DYS390 | 3.598 | 0.166 | 0.006 |
| DYS393 | DYS19 | 3.471 | 0.176 | 0.008 |
| DYS439 | DYS392 | 3.432 | 0.18 | 0.009 |
| DYS19 | DYS391 | 3.401 | 0.183 | 0.005 |
| DYS389I | DYS439 | 3.284 | 0.194 | 0.006 |
| DYS385 | DYS439 | 2.624 | 0.269 | 0.023 |
| DYS19 | DYS385 | 2.268 | 0.322 | 0.024 |
| DYS393 | DYS391 | 2.142 | 0.343 | 0.005 |
| DYS389II | DYS392 | 2.045 | 0.36 | 0.007 |
| DYS19 | DYS438 | 6.154 | 0.461 | 0.003 |
| DYS391 | DYS439 | 1.546 | 0.462 | 0.005 |
| DYS389II | DYS385 | 1.471 | 0.48 | 0.021 |
| DYS393 | DYS392 | 1.223 | 0.543 | 0.010 |
| DYS19 | DYS389II | 0.981 | 0.612 | 0.008 |
| DYS389II | DYS391 | 0.749 | 0.688 | 0.004 |
| DYS393 | DYS439 | 0.221 | 0.895 | 0.005 |

TABLE 10
Linkage disequilibrium analysis of
Y-PLEX loci in Caucasian population

| | | | | Standard |
|---|---|---|---|---|
| Locus 1 | Locus 2 | Chi2 | P-Value | Error |
| DYS390 | DYS438 | Infinity | Highly Significant | 0 |
| DYS385 | DYS438 | 17.748 | 0 | 0 |
| DYS439 | DYS438 | 13.353 | 0.001 | 0 |
| DYS391 | DYS438 | 12.802 | 0.002 | 0 |
| DYS438 | DYS392 | 11.27 | 0.004 | 0.001 |
| DYS389II | DYS389I | 11.183 | 0.004 | 0.001 |
| DYS393 | DYS385 | 10.857 | 0.004 | 0.002 |
| DYS389I | DYS392 | 10.821 | 0.004 | 0.001 |
| DYS389I | DYS438 | 10.366 | 0.006 | 0.001 |
| DYS391 | DYS385 | 10.328 | 0.006 | 0.001 |
| DYS390 | DYS439 | 9.159 | 0.01 | 0.002 |
| DYS390 | DYS392 | 8.626 | 0.013 | 0.002 |
| DYS391 | DYS392 | 8.1 | 0.017 | 0.001 |
| DYS390 | DYS389I | 7.773 | 0.021 | 0.001 |
| DYS385 | DYS389I | 7.308 | 0.026 | 0.003 |
| DYS390 | DYS391 | 7.045 | 0.03 | 0.001 |
| DYS19 | DYS439 | 6.61 | 0.037 | 0.004 |
| DYS19 | DYS392 | 6.516 | 0.038 | 0.003 |
| DYS390 | DYS385 | 5.624 | 0.06 | 0.005 |
| DYS393 | DYS438 | 5.285 | 0.071 | 0.003 |
| DYS389I | DYS439 | 5.229 | 0.073 | 0.004 |
| DYS19 | DYS390 | 5.038 | 0.081 | 0.004 |
| DYS19 | DYS438 | 4.538 | 0.103 | 0.005 |
| DYS393 | DYS389I | 4.415 | 0.11 | 0.003 |
| DYS385 | DYS392 | 3.877 | 0.144 | 0.009 |
| DYS393 | DYS391 | 3.771 | 0.152 | 0.003 |
| DYS391 | DYS389I | 3.694 | 0.158 | 0.002 |
| DYS439 | DYS392 | 3.646 | 0.162 | 0.007 |
| DYS19 | DYS389I | 2.583 | 0.275 | 0.007 |
| DYS389II | DYS392 | 2.089 | 0.352 | 0.006 |
| DYS389II | DYS438 | 2.01 | 0.366 | 0.008 |
| DYS391 | DYS439 | 1.84 | 0.399 | 0.004 |
| DYS385 | DYS439 | 1.489 | 0.475 | 0.017 |
| DYS393 | DYS392 | 1.283 | 0.526 | 0.006 |
| DYS389II | DYS439 | 0.973 | 0.615 | 0.008 |
| DYS393 | DYS19 | 0.915 | 0.633 | 0.007 |
| DYS19 | DYS391 | 0.685 | 0.71 | 0.003 |
| DYS389II | DYS385 | 0.676 | 0.713 | 0.011 |
| DYS389II | DYS390 | 0.614 | 0.736 | 0.005 |
| DYS389II | DYS391 | 0.581 | 0.748 | 0.003 |
| DYS393 | DYS439 | 0.552 | 0.759 | 0.005 |
| DYS19 | DYS389II | 0.445 | 0.8 | 0.005 |
| DYS19 | DYS385 | 0.359 | 0.835 | 0.013 |
| DYS393 | DYS390 | 0.168 | 0.92 | 0.002 |
| DYS393 | DYS389II | 0.159 | 0.924 | 0.002 |

TABLE 11
Linkage disequilibrium analysis of Y-PLEX
loci in African American population

| | | | | Standard |
|---|---|---|---|---|
| Locus 1 | Locus 2 | Chi2 | P-Value | Error |
| DYS438 | DYS392 | 8.159 | 0.017 | 0.001 |
| DYS393 | DYS389II | 7.163 | 0.028 | 0.003 |
| DYS391 | DYS392 | 5.38 | 0.068 | 0.001 |
| DYS389II | DYS390 | 4.777 | 0.092 | 0.005 |
| DYS390 | DYS389I | 4.638 | 0.098 | 0.002 |
| DYS393 | DYS385 | 4.261 | 0.119 | 0.007 |
| DYS385 | DYS438 | 4.204 | 0.122 | 0.007 |
| DYS19 | DYS392 | 4.059 | 0.131 | 0.003 |
| DYS393 | DYS390 | 3.964 | 0.138 | 0.005 |
| DYS390 | DYS438 | 3.887 | 0.143 | 0.006 |
| DYS19 | DYS389I | 3.245 | 0.197 | 0.004 |
| DYS391 | DYS438 | 3.217 | 0.2 | 0.004 |
| DYS19 | DYS439 | 3.187 | 0.203 | 0.006 |
| DYS390 | DYS392 | 2.532 | 0.282 | 0.003 |
| DYS393 | DYS438 | 2.531 | 0.282 | 0.007 |
| DYS393 | DYS389I | 2.403 | 0.301 | 0.004 |
| DYS389I | DYS438 | 2.373 | 0.305 | 0.004 |
| DYS389II | DYS392 | 1.97 | 0.373 | 0.004 |
| DYS389II | DYS391 | 1.605 | 0.448 | 0.005 |
| DYS19 | DYS390 | 1.418 | 0.492 | 0.008 |
| DYS19 | DYS438 | 1.403 | 0.496 | 0.009 |
| DYS390 | DYS385 | 1.298 | 0.522 | 0.012 |
| DYS385 | DYS392 | 1.251 | 0.535 | 0.005 |
| DYS390 | DYS391 | 1.157 | 0.561 | 0.003 |
| DYS389II | DYS439 | 1.025 | 0.599 | 0.006 |
| DYS393 | DYS19 | 0.889 | 0.641 | 0.008 |
| DYS393 | DYS392 | 0.854 | 0.652 | 0.003 |
| DYS19 | DYS391 | 0.81 | 0.667 | 0.005 |
| DYS389II | DYS385 | 0.783 | 0.676 | 0.011 |
| DYS393 | DYS391 | 0.606 | 0.739 | 0.003 |
| DYS19 | DYS385 | 0.458 | 0.795 | 0.01 |
| DYS385 | DYS439 | 0.404 | 0.817 | 0.007 |
| DYS19 | DYS389II | 0.348 | 0.84 | 0.006 |
| DYS390 | DYS439 | 0.305 | 0.858 | 0.004 |
| DYS389II | DYS438 | 0.177 | 0.915 | 0.004 |
| DYS393 | DYS439 | 0.173 | 0.917 | 0.003 |
| DYS391 | DYS385 | 0 | 1 | 0 |
| DYS389II | DYS389I | 0 | 1 | 0 |
| DYS391 | DYS389I | 0 | 1 | 0 |
| DYS385 | DYS389I | 0 | 1 | 0 |
| DYS391 | DYS439 | 0 | 1 | 0 |
| DYS389I | DYS439 | 0 | 1 | 0 |
| DYS439 | DYS438 | 0 | 1 | 0 |
| DYS389I | DYS392 | 0 | 1 | 0 |
| DYS439 | DYS392 | 0 | 1 | 0 |

TABLE 12
Linkage disequilibrium analysis of OSU
10-locus set in all 30 individuals.

| | | | | Standard |
|---|---|---|---|---|
| Locus 1 | Locus 2 | Chi² | P-value | Error |
| OSU14 | OSU09 | Infinity | Highly Significant | 0 |
| OSU73 | OSU70 | 14.237 | 0.00081 | 0.00032 |
| OSU73 | OSU09 | 11.458 | 0.00325 | 0.00143 |
| OSU14 | OSU77 | 7.607 | 0.02229 | 0.00701 |
| OSU09 | OSU70 | 7.122 | 0.02841 | 0.00468 |
| OSU57 | OSU70 | 6.501 | 0.03875 | 0.00737 |
| OSU14 | OSU73 | 6.189 | 0.04529 | 0.00756 |
| OSU22 | OSU70 | 5.748 | 0.05646 | 0.00546 |
| OSU35 | OSU57 | 5.466 | 0.06501 | 0.00927 |
| OSU57 | OSU73 | 5.248 | 0.07252 | 0.01020 |
| OSU22 | OSU51 | 4.417 | 0.10985 | 0.00802 |
| OSU22 | OSU35 | 4.275 | 0.11795 | 0.00784 |
| OSU77 | OSU09 | 4.151 | 0.12550 | 0.01317 |
| OSU35 | OSU73 | 4.134 | 0.12656 | 0.00835 |
| OSU35 | OSU77 | 3.689 | 0.15811 | 0.01001 |
| OSU35 | OSU67 | 3.547 | 0.16974 | 0.01470 |
| OSU22 | OSU73 | 3.395 | 0.18311 | 0.01007 |
| OSU35 | OSU09 | 3.198 | 0.20206 | 0.01568 |
| OSU35 | OSU70 | 3.110 | 0.21124 | 0.01023 |
| OSU51 | OSU70 | 3.016 | 0.22134 | 0.01263 |
| OSU67 | OSU77 | 2.989 | 0.22432 | 0.01408 |
| OSU14 | OSU67 | 2.938 | 0.23011 | 0.01666 |
| OSU57 | OSU09 | 2.899 | 0.23471 | 0.02111 |
| OSU22 | OSU67 | 2.625 | 0.26914 | 0.01202 |
| OSU22 | OSU57 | 2.561 | 0.27792 | 0.01404 |
| OSU35 | OSU51 | 2.411 | 0.29954 | 0.01654 |
| OSU67 | OSU09 | 2.367 | 0.30619 | 0.01679 |
| OSU22 | OSU77 | 2.263 | 0.32262 | 0.01286 |
| OSU51 | OSU73 | 2.159 | 0.33973 | 0.01638 |
| OSU14 | OSU70 | 2.059 | 0.35718 | 0.01583 |
| OSU67 | OSU70 | 2.001 | 0.36778 | 0.01211 |
| OSU77 | OSU70 | 1.980 | 0.37153 | 0.01347 |
| OSU22 | OSU09 | 1.843 | 0.3980 | 0.01205 |
| OSU51 | OSU57 | 1.755 | 0.41572 | 0.02496 |
| OSU14 | OSU22 | 1.468 | 0.48001 | 0.01494 |
| OSU51 | OSU67 | 1.442 | 0.48627 | 0.02060 |
| OSU57 | OSU67 | 1.280 | 0.52727 | 0.02158 |
| OSU73 | OSU77 | 1.156 | 0.56114 | 0.01471 |
| OSU14 | OSU57 | 0.869 | 0.64762 | 0.02591 |
| OSU67 | OSU73 | 0.598 | 0.74151 | 0.01458 |
| OSU57 | OSU77 | 0.464 | 0.79284 | 0.01481 |
| OSU14 | OSU51 | 0.446 | 0.80004 | 0.01627 |
| OSU51 | OSU09 | 0.191 | 0.90889 | 0.01021 |
| OSU14 | OSU35 | 0.177 | 0.9151 | 0.00897 |
| OSU51 | OSU77 | 0.092 | 0.95489 | 0.00491 |

TABLE 13
Linkage disequilibrium analysis of
OSU loci in Caucasian population

| | | | | Standard |
|---|---|---|---|---|
| Locus 1 | Locus 2 | Chi2 | P-value | Error |
| OSU67 | OSU70 | 8.804 | 0.012 | 0.002 |
| OSU67 | OSU73 | 7.885 | 0.019 | 0.003 |
| OSU73 | OSU70 | 7.57 | 0.023 | 0.002 |
| OSU14 | OSU9 | 5.639 | 0.06 | 0.008 |
| OSU14 | OSU77 | 5.452 | 0.065 | 0.009 |
| OSU57 | OSU70 | 4.407 | 0.11 | 0.014 |
| OSU22 | OSU70 | 3.533 | 0.171 | 0.006 |
| OSU22 | OSU57 | 3.476 | 0.176 | 0.011 |
| OSU51 | OSU70 | 3.291 | 0.193 | 0.012 |
| OSU77 | OSU9 | 2.9 | 0.235 | 0.011 |
| OSU14 | OSU22 | 2.608 | 0.271 | 0.01 |
| OSU35 | OSU51 | 2.424 | 0.298 | 0.014 |
| OSU67 | OSU77 | 2.029 | 0.363 | 0.012 |
| OSU57 | OSU77 | 2.001 | 0.368 | 0.018 |
| OSU35 | OSU67 | 1.965 | 0.374 | 0.016 |
| OSU9 | OSU70 | 1.755 | 0.416 | 0.012 |
| OSU35 | OSU77 | 1.617 | 0.446 | 0.013 |
| OSU51 | OSU73 | 1.561 | 0.458 | 0.007 |
| OSU35 | OSU57 | 1.535 | 0.464 | 0.02 |
| OSU51 | OSU9 | 1.394 | 0.498 | 0.013 |
| OSU22 | OSU35 | 1.306 | 0.52 | 0.008 |
| OSU67 | OSU9 | 1.248 | 0.536 | 0.015 |
| OSU14 | OSU57 | 1.105 | 0.576 | 0.024 |
| OSU22 | OSU77 | 1.004 | 0.605 | 0.008 |
| OSU73 | OSU77 | 0.914 | 0.633 | 0.007 |
| OSU73 | OSU9 | 0.896 | 0.639 | 0.007 |
| OSU57 | OSU73 | 0.779 | 0.677 | 0.01 |
| OSU57 | OSU67 | 0.775 | 0.679 | 0.019 |
| OSU35 | OSU73 | 0.74 | 0.691 | 0.007 |
| OSU22 | OSU9 | 0.671 | 0.715 | 0.007 |
| OSU57 | OSU9 | 0.491 | 0.782 | 0.015 |
| OSU14 | OSU51 | 0.485 | 0.785 | 0.014 |
| OSU14 | OSU70 | 0.469 | 0.791 | 0.014 |
| OSU14 | OSU67 | 0.387 | 0.824 | 0.013 |
| OSU77 | OSU70 | 0.345 | 0.841 | 0.009 |
| OSU35 | OSU9 | 0.33 | 0.848 | 0.008 |
| OSU51 | OSU57 | 0.273 | 0.873 | 0.012 |
| OSU14 | OSU73 | 0.269 | 0.874 | 0.006 |
| OSU35 | OSU70 | 0.266 | 0.876 | 0.008 |
| OSU22 | OSU73 | 0.245 | 0.885 | 0.003 |
| OSU22 | OSU51 | 0.149 | 0.928 | 0.003 |
| OSU51 | OSU67 | 0.118 | 0.943 | 0.006 |
| OSU51 | OSU77 | 0.01 | 0.995 | 0.001 |
| OSU14 | OSU35 | 0 | 1 | 0 |
| OSU22 | OSU67 | 0 | 1 | 0 |

TABLE 14
Linkage disequilibrium analyses of the OSU
loci in the African American population

| | | | | Standard |
|---|---|---|---|---|
| Locus 1 | Locus 2 | Chi2 | P-value | Error |
| OSU14 | OSU9 | 6.854 | 0.032 | 0.004 |
| OSU73 | OSU70 | 5.922 | 0.052 | 0.003 |
| OSU57 | OSU73 | 4.375 | 0.112 | 0.008 |
| OSU14 | OSU67 | 4.132 | 0.127 | 0.006 |
| OSU22 | OSU73 | 4.044 | 0.132 | 0.003 |
| OSU51 | OSU70 | 3.636 | 0.162 | 0.005 |
| OSU22 | OSU35 | 3.618 | 0.164 | 0.004 |
| OSU73 | OSU9 | 3.585 | 0.167 | 0.009 |
| OSU22 | OSU77 | 3.466 | 0.177 | 0.004 |
| OSU14 | OSU57 | 3.098 | 0.212 | 0.011 |
| OSU35 | OSU67 | 3.064 | 0.216 | 0.007 |
| OSU73 | OSU77 | 2.962 | 0.227 | 0.01 |
| OSU35 | OSU9 | 2.889 | 0.236 | 0.012 |
| OSU9 | OSU70 | 2.811 | 0.245 | 0.007 |
| OSU77 | OSU70 | 2.368 | 0.306 | 0.007 |
| OSU14 | OSU73 | 2.298 | 0.317 | 0.01 |
| OSU22 | OSU51 | 2.186 | 0.335 | 0.004 |
| OSU57 | OSU77 | 1.88 | 0.391 | 0.013 |
| OSU67 | OSU9 | 1.792 | 0.408 | 0.01 |
| OSU51 | OSU73 | 1.672 | 0.433 | 0.012 |
| OSU14 | OSU22 | 1.593 | 0.451 | 0.006 |
| OSU22 | OSU57 | 1.385 | 0.5 | 0.006 |
| OSU35 | OSU70 | 1.301 | 0.522 | 0.007 |
| OSU14 | OSU35 | 1.279 | 0.528 | 0.012 |
| OSU14 | OSU70 | 1.251 | 0.535 | 0.009 |
| OSU22 | OSU9 | 1.177 | 0.555 | 0.006 |
| OSU57 | OSU9 | 1.166 | 0.558 | 0.016 |
| OSU35 | OSU51 | 1.002 | 0.606 | 0.012 |
| OSU22 | OSU70 | 0.936 | 0.626 | 0.003 |
| OSU57 | OSU70 | 0.838 | 0.658 | 0.007 |
| OSU35 | OSU77 | 0.735 | 0.693 | 0.011 |
| OSU67 | OSU77 | 0.708 | 0.702 | 0.008 |
| OSU14 | OSU77 | 0.672 | 0.715 | 0.009 |
| OSU35 | OSU57 | 0.658 | 0.72 | 0.012 |
| OSU51 | OSU67 | 0.628 | 0.731 | 0.007 |
| OSU35 | OSU73 | 0.463 | 0.793 | 0.008 |
| OSU57 | OSU67 | 0.361 | 0.835 | 0.006 |
| OSU77 | OSU9 | 0.32 | 0.852 | 0.009 |
| OSU14 | OSU51 | 0 | 1 | 0 |
| OSU51 | OSU57 | 0 | 1 | 0 |
| OSU22 | OSU67 | 0 | 1 | 0 |
| OSU67 | OSU73 | 0 | 1 | 0 |
| OSU51 | OSU77 | 0 | 1 | 0 |
| OSU51 | OSU9 | 0 | 1 | 0 |
| OSU67 | OSU70 | 0 | 1 | 0 |

Assessment of the OSU set on the same population at a P-value of less than 0.01 identified three pairs of loci in linkage disequilibrium, and a P-value of less than 0.05 showed seven pairs of loci in linkage disequilibrium. Four loci were in linkage disequilibrium with at least one locus at a P-value of less than 0.01 while six loci were in linkage disequilibrium with at least one locus at a P-value of less than 0.05.

Linkage disequilibrium was also evaluated separately for the African American and Caucasian populations. The Hispanic and East Asian populations were eliminated from this portion of the analysis since only two individuals represent them. Separation of the African American and Caucasian population into two populations reduced the level of linkage disequilibrium for both sets of loci. Once again, the Y-PLEX loci showed higher linkage disequilibrium than the OSU set. Examination of the Caucasian population with the Y-PLEX loci revealed 10 pairs of loci in linkage disequilibrium at a P-value of less than 0.01 and 18 pairs of loci in linkage disequilibrium with a P-value of less than 0.05 (Table 10). Less linkage disequilibrium was seen in the African American population; no linkage disequilibrium was observed with a P-value of less than 0.01, and only two pairs of loci are in linkage disequilibrium with a P-value of less than 0.05 (Table 11). Assessment of the OSU set in the Caucasian population disclosed no pairs of loci in linkage disequilibrium with a P-value of less than 0.01 and three pairs of loci with a P-Value of less than 0.05 (Table 13). Again, the African American population displayed lower values of linkage disequilibrium; no pairs showed a level of significance at less than 0.01, and only one pair revealed a P-value of less than 0.05 (Table 14).

Table 15 correlates OSU numbers to D#S# numbers as described above.

TABLE 15
Correlation between OSU numbering system and D#S# numbering
system with accession ID noted.

| OSU# | D#S# | Accession ID |
|---|---|---|
| OSU6 | DYS653 | GDB: 11511416 |
| OSU9 | DYS657 | GDB: 11511424 |
| OSU10 | DYS656 | GDB: 11511422 |
| OSU11 | DYS658 | GDB: 11511428 |
| OSU12 | DYS453 | GDB: 11498119 |
| OSU13 | DYS659 | GDB: 11511430 |
| OSU14 | DYS660 | GDB: 11511432 |
| OSU15 | DYS661 | GDB: 11511434 |
| OSU16 | DYS662 | GDB: 11511436 |
| OSU20 | DYS663 | GDB: 11511438 |
| OSU21 | DYS664 | GDB: 11511440 |
| OSU22 | DYS665 | GDB: 11511442 |
| OSU23 | DYS666 | GDB: 11511444 |
| OSU24 | DYS667 | GDB: 11511446 |
| OSU25 | DYS668 | GDB: 11511448 |
| OSU26 | DYS669 | GDB: 11511450 |
| OSU27 | DYS655 | GDB: 11511420 |
| OSU28 | DYS670 | GDB: 11511452 |
| OSU31 | DYS671 | GDB: 11511454 |
| OSU32 | DYS455 | GDB: 11498125 |
| OSU33 | DYS672 | GDB: 11511456 |
| OSU34 | DYS673 | GDB: 11511458 |
| OSU35 | DYS674 | GDB: 11511460 |
| OSU37 | DYS675 | GDB: 11511462 |
| OSU38 | DYS676 | GDB: 11511464 |
| OSU40 | DYS677 | GDB: 11511466 |
| OSU42 | DYS678 | GDB: 11511468 |
| OSU43 | DYS679 | GDB: 11511470 |
| OSU44 | DYS680 | GDB: 11511472 |
| OSU45 | DYS681 | GDB: 11511474 |
| OSU46 | DYS463 | GDB: 11499418 |
| OSU47 | DYS682 | GDB: 11511476 |
| OSU48 | DYS683 | GDB: 11511478 |
| OSU49 | DYS684 | GDB: 11511480 |
| OSU50 | DYS654 | GDB: 11511417 |
| OSU51 | DYS685 | GDB: 11511482 |
| OSU52 | DYS458 | GDB: 11498131 |
| OSU53 | DYS686 | GDB: 11511484 |
| OSU54 | DYS687 | GDB: 11511486 |
| OSU55 | DYS449 | GDB: 10879367 |
| OSU56 | DYS454 | GDB: 11498123 |
| OSU57 | DYS688 | GDB: 11511488 |
| OSU58 | DYS689 | GDB: 11511490 |
| OSU59 | DYS690 | GDB: 11511492 |
| OSU60 | DYS691 | GDB: 11511494 |
| OSU61 | DYS692 | GDB: 11511496 |
| OSU62 | DYS693 | GDB: 11511498 |
| OSU63 | DYS694 | GDB: 11511500 |
| OSU64 | DYS695 | GDB: 11511502 |
| OSU65 | DYS696 | GDB: 11511504 |
| OSU66 | DYS697 | GDB: 11511506 |
| OSU67 | DYS698 | GDB: 11511508 |
| OSU68 | DYS699 | GDB: 11511510 |
| OSU69 | DYS700 | GDB: 11511512 |
| OSU70 | DYS448 | GDB: 10877524 |
| OSU71 | DYS701 | GDB: 11511514 |
| OSU72 | DYS702 | GDB: 11511516 |
| OSU73 | DYS703 | GDB: 11511518 |
| OSU74 | DYS704 | GDB: 11511520 |
| OSU75 | DYS705 | GDB: 11511522 |
| OSU76 | DYS706 | GDB: 11511524 |
| OSU77 | DYS707 | GDB: 11511526 |

### DOCUMENTS

The following documents, which form part of the disclosure of this application, are incorporated herein by reference.
Aaltonen L A, Peltomaki P, Leach F S, Sistonen P, Pylkkanen L, Mecklin J P, Jarvinen H, Powell S M, Jen J, Hamilton S R, Petersen G M, Kinzler K W, Vogelstein B, Delachapelle A (1993) Clues to the Pathogenesis of Familial Colorectal-Cancer. Science 260:812-816
Affara N A, Fergusin-Smith M A (1994) DNA sequence homology between the human sex chromosomes. In: Molecular genetics of sex determination. Academic Press Inc, pp 225-266
Agulnik A I, Mitchell M J, Lerner J L, Woods D R, Bishop C E (1994) A Mouse Y-Chromosome Gene Encoded by a Region Essential for Spermatogenesis and Expression of Male-Specific Minor Histocompatibility Antigens. Hum Mol Genet 3:873-878
Altschul S F, Gish W, Miller W, Myers E W, Lipman D J (1990) Basic Local Alignment Search Tool. J Mol Biol 215:403-410
Anslinger K, Keil W, Weichhold G, Eisenmenger W (2000) Y-chromosomal STR haplotypes in a population sample from Bavaria. Int J Legal Med 113:189-192
Atkin N B (2001) Microsatellite instability. Cytogenet Cell Genet 92:177-181
Austin J (1997) The Analysis of DNA Obtained from Hair Samples. Master of Science, The Ohio State University, Columbus
Ayub Q, Mohyuddin A, Qamar R, Mazhar K, Zerjal T, Mehdi S Q, Tyler-Smith C (2000) Identification and characterisation of novel human Y-chromosomal microsatellites from sequence database information. Nucleic Acids Research 28:e8
Benson G (1999) Tandem repeats finder: a program to analyze DNA sequences. Nucleic Acids Research 27:573-580
Blanco P, Sargent C A, Boucher C A, Howell G, Ross M, Affara N A (2001) A novel poly(A)-binding protein gene (PABPC5) maps to an X-specific subinterval in the Xq21.3/Yp11.2 homology block of the human sex chromosomes. Genomics 74:1-11
Bohossian H B, Skaletsky H, Page D C (2000) Unexpectedly similar rates of nucleotide substitution found in male and female hominids. Nature 406:622-625
Bosch E, Lee A C, Calafell F, Arroyo E, Henneman P, de Knijff P, Jobling Mass. (2002) High resolution Y chromosome typing: 19 STRs amplified in three multiplex reactions. Forensic Science International 125:42-51
Budowle B (2004) Understanding and Interpreting Y STR Evidence. Paper presented at Paper presented at Y-STR Analysis on Forensic Casework Workshop American Academy of Forensic Sciences 56^{th} Annual Meeting. Dallas, Tex., February 17
Butler J M, Schoske R, Vallone P M, Kline M C, Redd A J, Hammer M F (2002) A novel multiplex for simultaneous amplification of 20 Y chromosome STR markers. Forensic SciInt 129:10-24
Carracedo A, Beckmann A, Bengs A, Brinkman B, Caglia A, Capelli C, Gill P, et al. (2001) Results of a collaborative study of the EDNAP group regarding the reproducibility and robustness of the Y-chromosome STRs DYS19, DYS389 I and II, DYS390 and DYS393 in a PCR pentaplex format. Forensic Science International 119:28-41
Carvalho-Silva D R, Pena S. Dak. (2000) Molecular characterization and population study of an X chromosome homolog of the Y-linked microsatellite DYS391. Gene
Cohn D E, Basil J B, Venegoni A R, Mutch D G, Rader J S, Herzog T J, Gersell D J, Goodfellow P J (2000) Absence of PTEN repeat tract mutation in endometrial cancers with microsatellite instability. Gynecol Oncol 79:101-106
da Costa A N, Silva R, Moura-Neto R S (2002) Y-chromosome variation in a Rio de Janeiro, Brazil, population sample. Forensic SciInt 126:254-257
deknijff P, Kayser M, Caglia A, Corach D, Fretwell N, Gehrig C, Graziosi G, et al. (1997) Chromosome Y microsatellites: Population genetic and evolutionary aspects. International Journal of Legal Medicine 110:134-149
Delbridge M L, Lingenfelter P A, Disteche C M, Graves J A M (1999) The candidate spermatogenesis gene RBMY has a homologue on the human X chromosome. Nature Genet 22:223-224
Dieringer D, Schlotterer C (2003) Two distinct modes of microsatellite mutation processes: Evidence from the complete genomic sequences of nine species. Genome Res 13:2242-2251
Dolle J (2003) Characterization of Y-chromosome DNA microsatellite genetic markers in non-human primates. Senior Honors Thesis, The Ohio State University, Columbus
Duggan B D, Felix J C, Muderspach L I, Tourgeman D, Zheng J, Shibata D (1994) Microsatellite Instability in Sporadic Endometrial Carcinoma. J Natl Cancer Inst
Dupuy BM, Gedde-Dahl T, Olaisen B (2000) DXYS267: DYS393 and its X chromosome counterpart. Forensic Sci Int 112:111-21
Flint J, Boyce A J, Martinson J J, Clegg J B (1989) Population Bottlenecks in Polynesia Revealed by Minisatellites. Hum Genet 83:257-263
Foster J W, Brennan F E, Hampikian G K, Goodfellow P N, Sinclair A H, Lovellbadge R, Selwood L, Renfree M B, Cooper D W, Graves J A M (1992) Evolution of Sex Determination and the Y-Chromosome—Sry-Related Sequences in Marsupials. Nature 359:531-533
Foster J W, Graves J A M (1994) An Sry-Related Sequence on the Marsupial X-Chromosome—Implications for the Evolution of the Mammalian Testisdetermining Gene. Proc Natl Acad Sci USA 91:1927-1931
Geldwerth D, Bishop C, Guellaen G, Koenig M, Vergnaud G, Mandel J L, Weissenbach J (1985) Extensive DNA-Sequence Homologies between the Human-Y and the Long Arm of the X-Chromosome. Embo J 4:1739-1743
Gene M, Borrego N, Xifro A, Pique E, Moreno P, Huguet E (1999) Haplotype frequencies of eight Y-chromosome STR loci in Barcelona (North-East Spain). Int J Legal Med 112:403-405
Gill P, Brenner C, Brinkmann B, Budowle B, Carracedo A, Jobling M A, de Knijff P, Kayser M, Krawczak M, Mayr W R, Morling N, Olaisen B, Pascali V, Prinz M, Roewer L, Schneider P M, Sajantila A, Tyler-Smith C (2001) DNA Commission of the International Society of Forensic Genetics: recommendations on forensic analysis using Y-chromosome STRs. Int J Legal Med 114:305-309
Glaser B, Grutzner F, Taylor K, Schiebel K, Meroni G, Tsioupra K, Pasantes J, Rietschel W, Toder R, Willmann U, Zeitler S, Yen P, Ballabio A, Rappold G, Schempp W (1997) Comparative mapping of Xp22 genes in hominoids—Evolutionary linear instability of their Y homologues. Chromosome Res 5:167-176
Gonzalez-Neira A, Elmoznino M, Lareu MV, Sanchez-Diz P, Gusmao L, Mechthild P, Carracedo A (2001) Sequence structure of 12 novel Y chromosome microsatellites and PCR amplification strategies. Forensic Sci Int 122:19-26
Graves J A M, Wakefield M J, Toder R (1998) The origin and evolution of the pseudoautosomal regions of human sex chromosomes. Hum Mol Genet 7:1991
Gusmao L (2004) Y-STR's Allele Frequency Distributions Within Haplogroups. Paper presented at Paper presented at Y-STR Analysis on Forensic Casework Workshop American Academy of Forensic Sciences 56^{th} Annual Meeting. Dallas, Tex., February 17
Gusmao L, Alves C, Beleza S, Amorim A (2002a) Forensic evaluation and population data on the new Y-STRs DYS434, DYS437, DYS438, DYS439 and GATA A10. International Journal of Legal Medicine 116:139-147
Gusmao L, Gonzalez-Neira A, Alves C, Lareu M, Costa S, Amorim A, Carracedo A (2002b) Chimpanzee homologous of human Y specific STRs—A comparative study and a proposal for nomenclature. Forensic SciInt 126:129-136
Gusmao L, Gonzalez-Neira A, Pestoni C, Brion M, Lareu M V, Carracedo A (1999) Robustness of the YSTRs DYS19, DYS389 I and II, DYS390 and DYS393: optimization of a PCR pentaplex. Forensic Science International 106:163-172
Gusmao L, Gonzalez-Neira A, Sanchez-Diz P, Lareu M V, Amorim A, Carracedo A (2000) Alternative primers for DYS391 typing: advantages of their application to forensic genetics. Forensic Science International 112:49-57
Hou Y P, Zhang J, Li Y B, Wu J, Zhang S Z, Prinz M (2001) Allele sequences of six new Y-STR loci and haplotypes in the Chinese Han population. Forensic SciInt 118:147-152
Hurles M E, Irven C, Nicholson J, Taylor P G, Santos F R, Loughlin J, Jobling M A, Sykes B C (1998) European y-chromosomal lineages in Polynesians: A contrast to the population structure revealed by mtDNA. Am J Hum Genet 63:1793-1806
Iida R, Tsubota E, Matsuki T (2001) Identification and characterization of two novel human polymorphic STRs on the Y chromosome. Int J Legal Med 115:54-6
Iida R, Tsubota E, Sawazaki K, Masuyama M, Matsuki T, Yasuda T, Kishi K (2002) Characterization and haplotype analysis of the polymorphic Y-STRs DYS443, DYS444 and DYS445 in a Japanese population. Int J Legal Med 116:191-194
Jegalian K, Page D C. (1998) A proposed path by which genes common to mammalian X and Y chromosomes evolve to become X inactivated. Nature 394:776-780
Kayser M, Caglia A, Corach D, Fretwell N, Gehrig C, Graziosi G, Heidorn F, et al. (1997) Evaluation of Y-chromosomal STRs: a multicenter study. Int J Legal Med 110:125-33, 141-9
Kayser, M., Kittler, R., Erler, A., Hedman, M., Lee, A. C., Mohyuddin, A., Mehdi, S. Q., Rosser, Z., Stoneking, M., Jobling, M. A., Sajantila, A. & Tyler-Smith, C. (2004) A comprehensive survey of human Y-chromosomal microsatellites. Am. J. Hum. Genet. 74, 1183-1197.
Kobayashi K, Sagae S, Kudo R, Saito H, Koi S, Nakamura Y (1995) Microsatellite Instability in Endometrial Carcinomas—Frequent Replication Errors in Tumors of Early-Onset and/or of Poorly Differentiated Type. Gene Chromosomes Cancer 14:128-132
Lambson B, Affara N A, Mitchell M, Ferguson-Smith M A (1992) Evolution of DNA sequence homologies between the sex chromosomes in primate species. Genomics 14:1032-1040
Lessig R, Edelmann J (2001) Population data of Y-chromosomal STRs in Lithuanian, Latvian and Estonian males. Forensic SciInt 120:223-225
Li W-H (1997) Gene Structure, Genetic Codes, and Mutation. In: Molecular Evolution. Sinauer Associates Inc., Sunderland, pp 7-34
Lum J K, Rickards O, Ching C, Cann R L (1994) Polynesian Mitochondrial Dnas Reveal 3 Deep Maternal Lineage Clusters. Hum Biol 66:567-590
Mumm S, Molini B, Terrell J, Srivastava A, Schlessinger D (1997) Evolutionary features of the 4-Mb Xq21.3 XY homology region revealed by a map at 60-kb resolution. Genome Res 7:307-314
Nishizawa M, Nishizawa K (2002) A DNA sequence evolution analysis generalized by simulation and the Markov chain Monte Carlo method implicates strand slippage in a majority of insertions and deletions. J Mol Evol 55:706-717
Ohno S (1967) Sex chromosomes and sex linked genes. In: Londhardt A (ed) Monographs on Endocrinology. Springer-Verlag, New York
Page D C, Harper M E, Love J, Botstein D (1984) Occurrence of a Transposition from the X-Chromosome Long Arm to the Y-Chromosome Short Arm During Human-Evolution. Nature 311:119-123
Parreira K S, Lareu M V, Sanchez-Diz P, Skitsa I, Carracedo A (2002) DNA typing of short tandem repeat loci on Y-chromosome of Greek population. Forensic Science International 126:261-264
Peiffer S L, Herzog T J, Tribune D J, Mutch D G, Gersell D J, Goodfellow P J (1995) Allelic Loss of Sequences from the Long Arm of Chromosome-10 and Replication Errors in Endometrial Cancers. Cancer Res 55:1922-1926
Perez-Lezaun A, Calafell F, Comas D, Mateu E, Bosch E, Martinez-Arias R, Clarimon J, Fiori G, Luiselli D, Facchini F, Pettener D, Bertranpetit J (1999) Sex-specific migration patterns in central Asian populations, revealed by analysis of Y-chromosome short tandem repeats and mtDNA. American Journal of Human Genetics 65:208-219
Redd A J, Agellon A B, Kearney V A, Contreras V A, Karafet T, Park H, de Knijff P, Butler J M, Hammer M F (2002) Forensic value of 14 novel STRs on the human Y chromosome. Forensic SciInt 130:97-111
Ricci U, Sani I, Uzielli M L G (2001) Y-chromosomal STR haplotype in Toscany (central Italy). Forensic SciInt 120:210-212
Risinger J I, Berchuck A, Kohler M F, Watson P, Lynch H T, Boyd J (1993) Genetic Instability of Microsatellites in Endometrial Carcinoma. Cancer Res 53:5100-5103
Rohlf J F, Sokal R R (1969) Statistical Tables. In: Emerson R (ed) Biometry. W.H. Freeman and Company, San Francisco, pp 1-253
Roy C M, Miller Coyle H, Hintz J L, Neylon S, Ladd C, Lee H C (2002) A Validation Study of Y-PLEX™ 6 a Multiplexed Y-Chromosome STR System. Paper presented at American Academy of Forensic Sciences 54th Annual Meeting. Atlanta, Ga., February 11-16
Rozen S, Skaletsky H J (2000) Primer3 on the WWW for general users and for biologist programmers. In: Krawetz S M S (ed) Bioinformatics Methods and Protocols: Methods in Molecular Biology. Humana Press, Totowa, pp 365-386
Sargent C A, Boucher C A, Blanco P, Chalmers I J, Highet L, Hall N, Ross N, Crow T, Affara NA (2001) Characterization of the human Xq21.3/Yp11 homology block and conservation of organization in primates. Genomics 73:77-85
Schlotterer C (2000) Evolutionary dynamics of microsatellite DNA. Chromosoma
Schwartz A, Chan D C, Brown L G, Alagappan R, Peftay D, Disteche C, McGillivray B, de la Chapelle A, Page D C. (1998) Reconstructing hominid Y evolution: X-homologous block, created by X-Y transposition, was disrupted by Yp inversion through LINE-LINE recombination. Hum Mol Genet 7:1-111
Shin D K, Jin H J, Kwak K D, Choi J W, Han M S, Kang P W, Choi S K, Kim W (2001) Y-Chromosome multiplexes and their potential for the DNA profiling of Koreans. International Journal of Legal Medicine 115:109-117
Skaletsky H, Kuroda-Kawaguchi T, Minx P J, Cordum H S, Hillier L, Brown L G, Repping S, et al. (2003) The male-specific region of the human Y chromosome is a mosaic of discrete sequence classes. Nature 423:825-U2
Strachan T, Read A P (2004) Molecular Pathology. In: Kingston F (ed) Human Molecular Genetics. Vol 3. Garland Publishing, New York, pp 462-485
Strand M, Prolla T A, Liskay R M, Petes T D (1993) Destabilization of Tracts of Simple Repetitive DNA in Yeast by Mutations Affecting DNA Mismatch Repair. Nature
Sykes B, Leiboff A, Lowbeer J, Tetzner S, Richards M (1995) The Origins of the Polynesians—an Interpretation from Mitochondrial Lineage Analysis. Am J Hum Genet 57:1463-1475
Tatusova T A, Madden T L (1999) BLAST 2 SEQUENCES, a new tool for comparing protein and nucleotide sequences. FEMS Microbiol Lett 174:247-250
Thompson J D, Gibson T J, Plewniak F, Jeanmougin F, Higgins D G (1997) The CLUSTAL_X windows interface: flexible strategies for multiple sequence alignment aided by quality analysis tools. Nucleic Acids Research 25:4876-4882
Trovoada M J, Alves C, Gusmao L, Abade A, Amorim A, Prata M J (2001) Evidence for population sub-structuring in Sao Tome e Principe as inferred from Y-chromosome STR analysis. Ann Hum Genet 65:271-283
Waters P D, Duffy B, Frost C J, Delbridge M L, Graves J A M (2001) The human Y chromosome derives largely from a single autosomal region added to the sex chromosomes 80-130 million years ago. Cytogenetics and Cell Genetics 92:74
White P S, Tatum O L, Deaven L L, Longmire J L (1999) New, male-specific microsatellite markers from the human Y chromosome. Genomics 57:433-7
Wierdl M, Dominska M, Petes T D (1997) Microsatellite instability in yeast: Dependence on the length of the microsatellite. Genetics 146:769-779
Wilson M R, Polanskey D, Butler J, Dizinno J A, Replogle J, Budowle B (1995) Extraction, Pcr Amplification and Sequencing of Mitochondrial-DNA from Human Hair Shafts. Biotechniques 18:662-&
Wu F C, Pu C E (2001) Multiplex DNA typing of short tandem repeat loci on Y chromosome of Chinese population in Taiwan. Forensic SciInt 120:213-222
Zaharova B, Andonova S, Gilissen A, Cassiman J J, Decorte R, Kremensky 1 (2001) Y-chromosomal STR haplotypes in three major population groups in Bulgaria. Forensic SciInt 124:182-186
Zhu Y, Strassmann J E, Queller D C (2000) Insertions, substitutions, and the origin of microsatellites. Genet Res 76:227-236

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A DNA amplification primer pair for the amplification of at least one STR marker, wherein the primer pair is chosen from the primer pairs listed in Table 4.

2. The DNA amplification primer pair according to claim 1, wherein the primer pair is chosen from the primer pairs corresponding to those loci listed in Table 5.

3. A method for DNA fingerprinting at least one genetically related or unrelated individual, comprising:
a) exposing a DNA sample of an individual to at least one primer specific for a Y chromosome polymorphism at a predetermined loci, said loci being chosen from those listed in Table 2, with the proviso that if any of the “Redd” loci listed in Table 5 is selected then at least one other non-“Redd” locus from Table 2 is also selected;
b) amplifying DNA of the DNA sample using the at least one primer specific for a Y chromosome polymorphism; and
c) identifying the size of an amplified product.

4. The method according to claim 3, wherein the DNA amplification of step b) is effected by PCR or by asymmetric PCR procedure.

5. The method according to claim 4, wherein the amplifying is performed using a primer pair according to claim 1.

6. A method for DNA fingerprinting identification of human DNA samples, comprising:
a) exposing a DNA sample of an individual to at least one primer specific for a Y chromosome polymorphism at a predetermined loci, said loci being chosen from OSU9, OSU14, OSU22, OSU35, OSU51, OSU57, OSU67, OSU70, OSU73, OSU77, with the proviso that if OSU70 is selected then at least one other OSU locus is also selected;
b) amplifying DNA of the DNA sample using the at least one primer specific for a Y chromosome polymorphism; and
c) identifying the size of an amplified product.

7. The method according to claim 6, wherein said DNA fingerprinting of said DNA samples is for verifying transplanted tissues in research or therapeutic procedures.

8. The method according to claim 6, wherein said DNA fingerprinting of said DNA samples is for single cell genetic profiling in research or therapeutic procedure.

9. The method according to claim 6, wherein said DNA fingerprinting of said DNA samples is for verifying sample mix-up or contamination.

10. The method according to claim 6, wherein said DNA fingerprinting of said DNA samples is for testing, establishing or verifying paternity, maternity or consanguinity of individuals.

11. A kit for amplification of Y chromosomal polymorphisms, comprising:
a) at least one primer pair according to claim 1;
b) at least one reagent necessary for carrying out DNA amplification; and
c) at least one component that makes it possible to determine length of an amplified fragment.

12. The kit according to claim 11, further comprising at least one of a positive control and a negative control.

13. A method for determining the degree of relatedness between two or more individuals having the same or a different surname, comprising:
a) obtaining a DNA sample from said individuals;
b) amplifying said DNA by polymerase chain reaction using primers specific for Y chromosome polymorphisms at predetermined loci, said loci being selected from the group consisting of OSU9, OSU14, OSU22, OSU35, OSU51, OSU57, OSU67, OSU70, OSU73, OSU77, with the proviso that if OSU70 is selected then at least one other OSU locus is also selected;
c) determining the haplotypes of said individuals; and
d) comparing said haplotypes across a plurality of predetermined loci to determine the degree of relatedness between said individuals.

14. The method as claimed in claim 13, wherein said DNA sample is isolated from a source chosen from of blood cells, fingernail slices, hair follicles, sperm cells, buccal cells, bone cells, bone marrow cells, teeth, and epithelial cells.
